(19) United States
(12) Patent Application Publication
(10) Pub. No.: US 2003/0040129 A1
(43) Pub. Date: Feb. 27, 2003

(54) Binding assays using magnetically immobilized arrays

(76) Inventor: Haresh P. Shah, Santa Clara, CA (US)

Correspondence Address: Ray K. Shahani, Esq., ATTORNEY AT LAW, Twin Oaks Office Plaza, 477 Ninth Avenue, Suite 112, San Mateo, CA, 94402-1854, US

(21) Appl. No.: 10/206,721

(22) Filed: Jul. 26, 2002

Related U.S. Application Data
(60) Provisional application No. 60/313,341, filed on Aug. 20, 2001.

(51) Int. Cl. G01N 33/553

(52) U.S. Cl. 436/526

(57) ABSTRACT

Systems and methods for preparing and using magnetic particle arrays are provided. In conventional assays, a target molecule is immobilized in a particular array position on the surface of the substrate by chemically conjugating the molecule to the surface of the substrate. According to embodiments of the present invention, target biomolecules are immobilized magnetically rather than chemically. Accordingly, the target molecules are chemically conjugated to the surface of a magnetic particle, and it is the magnetic particles that are positioned in an array by printing (or spotting) the magnetic particles onto the surface of a magnetic array substrate. The array is exposed to a solution of probe molecules (analytes) having detector labels, and the positions in the array where complementary target binds to probe are recorded. Such magnetic particle arrays may be used in a variety of applications, including drug screening, nucleic acid sequencing, mutation analysis, medical diagnosis, and immunoassay analysis. The magnetic particle array may be The advantages of a magnetic particle array, which may be configured with a holder having microfluidic channels to deliver sample to the array to comprise a magnetic array biochip, include reduced assay variability, enhanced flexibility, lower cost and higher throughput.

### RELATED APPLICATION(S)

This application is related to Provisional Patent Application Serial No. 60/313,341 filed on Aug. 20, 2001 and claims any and all benefit of priority of filing date of said Provisional Application as may be entitled to thereby.

### BACKGROUND

1. Field of the Invention

The present invention generally relates to biochip microarrays for performing multiple, high throughput, biological and chemical assays. In particular, the present invention is directed toward novel methods of making and using microarrays with magnetic particles to immobilize biomolecular targets onto a substrate in a spatially defined, and physically addressable manner.

2. Description of the related art

Many methods for simultaneously analyzing multiple analytes in a given sample have been devised, and these are widely used in the fields of molecular biology, genomics, proteomics, pharmacology, combinatorial chemistry, and clinical diagnostics. Multiplexed assays of biomolecules are now a mainstay of these fields. Efforts by biopharmaceutical and academic laboratories to screen very large numbers of synthetic, natural, or recombinant compound libraries have inspired the development of new technologies suitable for rapid quantification and high-throughput screening.

Microarrays of binding agents such as proteins, cells, oligonucleotides, and polynucleotides have become increasingly important tools in biotechnology. These binding agent microarrays, in which a plurality of binding agents are deposited onto a solid substrate surface in the form of an array or pattern, find use in a variety of applications, including drug screening, nucleic acid sequencing, mutation analysis, medical diagnostics, and immunoassay analyses.

A critical feature of a typical positional microarray is that each of the biomolecules of the array is stably attached to a discrete location on a substrate surface, such that the position of the attached biomolecule remains constant and known throughout the assay. Generally, the biomolecules of a microarray are bound either directly to the substrate, or indirectly to the substrate through a linking group. Currently the most commonly used substrate is glass, but more recently, polyacrylamide and polyurethane based polymers have been employed as a substrate materials.

FIGS. 1A-1D illustrate a microarray according to techniques generally known in the art. Referring to FIG. 1A, samples are removed from a microtiter well plate (not shown) and deposited onto a substrate generally indicated at 100. Printed (or spotted) onto substrate 100 are samples arranged in an array format where the positions of the array may be designated by the labels A1, A2, A3, A4, B1, B2, B3, etc., where the letter identifies a column and the number a row. The purpose of the assay in this exemplary case is to determine whether or not a particular probe is a complementary match to a target sample. Samples are deposited onto substrate 100 with positional information (and thus their identities) preserved.

In FIG. 1B, a sample (which may be a sequence of messenger RNA, for example), is chemically conjugated to the surface of the substrate by a covalent bond 106 at position B3. Next, a probe 108 with an attached label 110 is exposed to each of the target samples at chosen positions of the array, as shown in FIG. 1C at the position B3. The probe 108 may be a sequence of DNA, which will bind to the mRNA target sample at location B3 if the mRNA has a sequence of nucleotides complementary to those of the DNA probe 108. The hybridization reaction is shown schematically at 112 in FIG. 1D. Excess probe is then washed off the substrate, leaving only probe that is bound to targets. The assay may then be imaged for the presence of the label 110 to determine which of the samples are a positive match. Again, it should be emphasized that each biomolecule target 104 is immobilized individually on the surface of substrate 100 by chemical conjugation at 106.

The basic approaches for immobilizing the biomolecule of interest onto a solid substrate in a defined pattern (the array) using chemical conjugation fall into two general categories. In the first such approach, the biomolecules are directly synthesized on the array support, while in second approach biomolecules are attached to the support post-synthetically. Each approach has its limitations. For example, when an array is created by direct synthesis on an array support, the efficiency of each synthetic step affects the quality and integrity of the biomolecules involved, potentially resulting in an undesirable percentage of incorrectly synthesized molecules and incomplete sequences. Such contaminants can interfere with subsequent use of the array. In contrast, the second approach to array production allows the desired molecules to be synthesized and purified by conventional methods prior to their formation into an array. Consequently, the quality of arrayed molecules, and thus the quality of the resultant array, is potentially greater with the second approach than the first.

An example of the first approach to chemical conjugation employs light, and a series of photo-lithographic masks to activate specific sites on a substrate, such as derivatized glass, in order to selectively bind nucleic acids thereto and, subsequently, attach additional nucleic acids to form known oligonucleotides at the desired locations. Unfortunately, these biochips are very expensive to produce, requiring photolithographic equipment; multiple steps and lengthy to incubation/washing times during manufacture, and are generally limited to relatively short nucleic acid samples (e.g., less than 20 base pairs). An example of the second approach is the transfer of pre-synthesized molecules by a variety of non-contact “ink jet” dispensers, such as piezoelectric and syringe-solenoid devices, or contact printing dispensers, such as microspotting pins, spit pins, quills, or tweezers.

In addition to the above-mentioned techniques used in affinity binding assays, there are separation techniques that do not involve chemically conjugating a biomolecule to the surface of the substrate supporting the array. One such technology involves the use of magnetic particles, whereby biomolecules in solution are bound to the surfaces of magnetic particles suspended in the solution, after which the magnetic particles are collected by the application of an external magnetic field. Such magnetic separations have been employed to sort cells, to recover antibodies or enzymes from solutions, to purify proteins using affinity techniques, and to remove unwanted particles from suspensions. These particles are used in separation processes as an alternative to centrifugation and filtration because the separation is rapid, and because the particles may be customized to a variety of different assays.

A schematic diagram of an exemplary magnetic separation technique is shown in FIGS. 2A-2C. In FIG. 2A, micron-sized magnetic particles 202 are coated with a polymeric layer (not shown) in order to chemically conjugate biomolecules 204 to their surfaces. In FIG. 2B, the magnetic particles 202, with their attached biomolecules 204, are suspended in a fluid medium 206 contained within an assay tube 208. In FIG. 2C, a magnetic field from an external magnet 210 may be used to separate the magnetic particles 202 from the fluid medium 206. However, the disclosed separation devices and methods for magnetic particles cannot be applied very well to high throughput multiplexed applications in which arrays of discrete magnetic particles are required.

Microarray can be divided broadly into two formats: 1) Positional Microarrays in which the arrays are fixed in a spatially defined and physically addressable manner onto the surface of the substrate before the microarray experiments are performed and 2) Random (virtual) Array formats in which arrays are not positionally fixed and their position remains variable throughout the experiment. Spotted and synthesized arrays on a slide or chip are examples of positional arrays whereas bead arrays are the example of virtual arrays.

Recently random (virtual) arrays employing beads are described. In bead arrays position of the at any time during the test is unknown and remains variable, This is in contrast to positional arrays in which position of the array is fixed to the surface of the substrate and the position of the probe on the surface serves to identify the probe (e.g. oligonucleotide array on a glass slide). In bead arrays, the position of the bead is variable (random) and for this reason it is necessary to encode each bead with unique tags (identification or detectable marker) to identify the attached probe. Furthermore decoding of individual bead requires using special instruments at the end of the test to identify probe and to analyze the results. Bead based arrays offer advantages in chemical flexibility, rapid turnaround and improved signal to noise ratio.

U.S. Pat. No. 2002/0081714 published Jun. 27, 2002 and filed Aug. 7 2001 by Jain et al., describes random (virtual) bead array using magnetic bead. In this method, position of the bead remains random and variable throughout assay and hence each magnetic bead is encoded using variety of methods prior to its use. However, to avoid special instrumentation, authors have devised a magnetic substrate to trap individual bead(s). In this method magnetic bead is trapped singly on the surface of the substrate at the end of the assay test and then read by conventional detection methods (e.g., CCD imaging). Geometries (dimensions) of magnetic trapping region is very critical in this method. Since individual bead is trapped, length, width and height of each magnetic region is carefully controlled and optimized to avoid trapping of two or more magnetic beads at the each magnetic region. In this approach, size of the magnetic capture region is approximately equal to the size of the magnetic bead used for the assay and the substrate is fabricated using complex photolithographic techniques. Magnetic chip (substrate) design varies with the size of the magnetic bead used for the assay. Magnetic field strength of each magnetic region is optimized to capture single bead. Any variation in magnetic strength and in dimension of magnetic region may lead to trapping more than one bead per magnetic region, which results in erroneous results. Furthermore, variation in magnetic bead size also leads to sub-optimal quality of results. Reagent containing plurality of encoded beads are dispensed on the surface of the array substrate and allowed to trap individually on the magnetic region. Gentle washing of the chip washes away the immobilized beads and chip can be reused. Compared to conventional bead arrays , this method eliminates the need for a special instrumentation (e.g., flow cytometers) for the analysis. FIG. 18 A is a representative of flowchart showing the assay method of the prior art.

Each of the above mentioned approaches contain inherent limitations in that they depend either on expensive and intensive photolithographic techniques, elaborate synthetic multi-step chemical schemes, or complex substrate chemistries. Those prior art approaches that utilize magnetic particles are not able to preserve positional information. There is a need in the industry for a simple, cost effective, flexible, and high-throughput method for constructing a reliable and stable multi-functional microarray. Improved method should be efficient, versatile, and capable of providing the stable attachment of a biomolecule to a position within the microarray. More particularly, what is needed is a simple method of immobilizing a biomolecule in an array position that is not dependent upon the chemistry of the array surface.

### SUMMARY OF THE INVENTION

In accordance with the subject invention, systems and subassemblies are provided for performing multiplexed interactions between molecules on a plurality of magnetic particles. The magnetic particles to which different entities are bound are distributed on a surface having individually magnetic sites, immobilized to specific sites by the magnetic field of the magnet layer. The spatial organization of the magnetic sites is preferably selected to be spaced in accordance with the spatial organization of a plurality of vessels that can serve as the magnetic particles, where each vessel defines the nature of the particle and, thus, the site of the particle(s). Employing battery of magnetic particle transfer units having the same spatial organization of a plurality of the vessels, particles are transferred to the magnet layer in the same spatial organization as the vessels. With the magnetic sites being more densely spaced than the vessels, by repetitively transferring magnetic particle from the same or different vessels, while placing the transfer units at successive positions in relation to the rows and columns of the magnetic sites, if desired, all the magnetic sites can be occupied with magnetic particles, with each site defined as to the nature of the occupying particle.

The system can include data processing so as to monitor the nature of the particles, magnetic sites occupied by individual or group of related particles, and the results of any processing of the particles while bound to the magnetic layer.

After the magnetic particles have been distributed onto the magnetic layer, the particles may be processed by adding one or more liquids that may derived from a sample, reagents etc., where the molecules on the particle may react with the components of the liquid.

Depending upon the nature of the process, the particles may be interrogated while bound to the layer or individually interrogated to determine the results of the processing.

Aspects of the present invention provide systems and methods for using magnetic particle arrays in a chemical or biological assay to detect the presence of an analyte in a given sample. Magnetic particle arrays and assays may be used in the fields of biology, genomics, proteomics, pharmacology, combinatorial chemistry, and clinical diagnostics. In conventional methods of preparing a bioassay, a target biomolecule is chemically conjugated to the surface of an array substrate, and a probe molecule having an attached marker is flowed onto the array wherein the probe then binds to the target at positions where the probe is chemically compatible or complementary. In contrast, with magnetic particle arrays the target biomolecule is conjugated to the surface of a magnetic particle, the magnetic particle is stably positioned in an array magnetically, and the assay is continued as in conventional techniques. Magnetic immobilization of the target biomolecules has many potential advantages over immobilizing the target biomolecules by chemical means.

In general, magnetic particles offer advantages that include ease and speed of handling, rapid reaction kinetics, convenience, low cost, and the large surface area available for biomolecular immobilization on the surfaces of the magnetic particles. Magnetic particles can be used with both aqueous and non-aqueous based solvents, and can be easily conjugated to the biomolecules of interest. For example, magnetic particles are particularly useful in heterogeneous binding assays as a solid phase reagent in immunoassays and DNA probe assays.

The magnetic array substrate of the present invention comprises a magnet layer, optionally supported by an array support layer, bonded to, or otherwise associated with a magnetic layer, the former which provides the source of a magnetic field that attracts the magnetic particles toward the array support layer. The combined assembly of the array support layer and the magnetic layer comprise a magnetic array substrate. The magnetic layer or the magnetic array substrate may be inserted into a holder to complete a magnetic biochip. The holder serves several functions, including a means in which the microfluidic components of the biochip are used to supply a liquid composition, e.g.,chemical reagent (which may be called the probe) to the array. The magnetic array substrate holds the magnetic particles with their immobilized molecules in a particular positional or spatial arrangement, such that the binding or complementary nature of the probe at specific spots on the array may be determined.

The composition of the array support layer is not critical to the invention and may be fabricated from a variety of materials, including metals, glass, gels, polymers, or semiconductors. The magnetic layer is a critical component of the magnetic array substrate because it generates the magnetic field that attracts the magnetic particles onto the surface of the magnetic array substrate. The array support layer may be physically bonded to the magnetic layer, using an adhesive for example, or it may be mechanically clamped using fasteners, clips, brackets, or the like. The magnetic array substrate may be incorporated into a biochip device that provides auxiliary structures such as microfluidic capillary channels for transporting sample fluids to and from the array. Additionally, the biochip may be an integral part of a larger system that includes controllers, pumping devices, imaging systems, and assorted support devices.

The magnetic particles of the present invention are understood to encompass magnetic beads, magnetic spheres, microclusters, or any type of magnetically responsive particle. The magnetic particles may have a core of magnetic material coated with a polymeric shell whose surfaces comprises the functional groups that provide the conjugating chemistry for attaching to a biomolecule. Alternatively, the magnetic particle may comprise a polymeric matrix into which is impregnated a small amount of a paramagnetic or ferromagnetic substance. The magnetic particles may have a wide range of diameters. The attachment of the biomolecules to the magnetic particles may be accomplished by covalent and/or ionic binding, by physical adsorption, or by affinity binding. A wide variety of functional groups are available including hydroxyl, carboxyl, cyano, mercapto, ethylene, thiol, amino, aldehyde groups, and the like.

Conventional magnetic transfer devices, e.g., micropipettes, may be used to spot the magnetic particles onto an array substrate. A small volume of fluid containing the magnetic particles is loaded into the tip of the pipette, and the fluid containing the magnetic particles is then dispensed onto the surface of the magnetic array substrate at predetermined locations. In contrast to conventional micropipetting techniques, the use of an electromagnetic pin and or magnetic transfer probe is extraordinarily well suited for spotting magnetic particles arrays.

In comparison to prior art, the present magnetic particle array is a positional array in which magnetic beads are immobilized in a spatially defined and physically addressable manner on the magnet layer. Magnetic beads are positionally dispensed at the predetermined locations on the magnet layer using robotic dispensing/spotting or transfer system prior the microarray experiments. Location of the beads carrying probe is known throughout the microarray experiment and hence do not require special coding and decoding, or special instruments to analyze the results.

The advantages of the magnetic particle arrays of the present invention include reduced assay variability, enhanced assay flexibility, and greater assay throughput.

Numerous other advantages and features of the present invention will become readily apparent from the following detailed description of the invention and the embodiments thereof, from the claims and from the accompanying drawings.

### BRIEF DESCRIPTION OF THE DRAWINGS

FIGS. 1A-1D represent a schematic diagram of a prior art microarray where target biomolecules are chemically conjugated to the surface of an array support layer.

FIGS. 2A-2C represent a schematic diagram of a prior art assay that uses an external magnet to collect magnetic particles from a suspension.

FIGS. 3A-3C illustrate an exemplary array support layer, magnetic layer, magnetic particle(s), magnetic array support, and holder with microfluidic channel(s) that comprise a magnetic array biochip of the present invention.

FIGS. 4A-4B and 5A-5B illustrate assays according to embodiments of the present invention using magnetic particles: the immobilized target biomolecules are nucleotide sequences and immunoglobulins in FIGS. 4 and 5, respectively.

FIGS. 6A-6C illustrate exemplary methods for attaching or bonding an array support layer to a magnetic layer.

FIGS. 7A-7B illustrate an assembled biochip device.

FIGS. 8A-8C illustrate methods by which microfluidic channels may be incorporated into the biochip device.

FIG. 9 illustrates an alternative way of constructing the microfluidic portion of the biochip.

FIG. 10 illustrates exemplary commercial embodiments of the magnetic biochips of the present invention.

FIGS. 11 and 12A-12B illustrate an exemplary embodiment of the present invention that uses a set of electromagnetic pins to print the magnetic particle array.

FIGS. 12C illustrate an exemplary embodiment of the present invention that uses magnetic transfer probe(s) a to print the magnetic particle array.

FIGS. 13A-D and 14 illustrate the ability of embodiments of the present invention to provide reduced assay variability relative to a conventional assay.

FIGS. 15A-B and 16 illustrate the ability of embodiments of the present invention to provide enhanced flexibility relative to a conventional assay.

FIG. 17 illustrates the examples of multi-pole magnetic patterns for the magnet layer.

FIG. 18A is a representative flowchart showing a method of the prior art.

FIG. 18B is a representative flowchart showing a preferred embodiment of a method of the present invention.

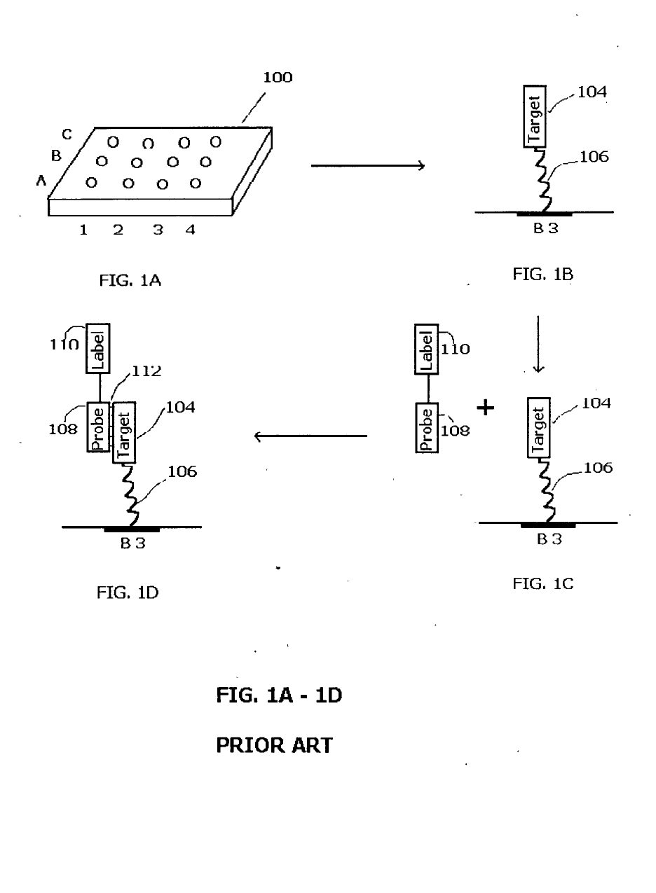

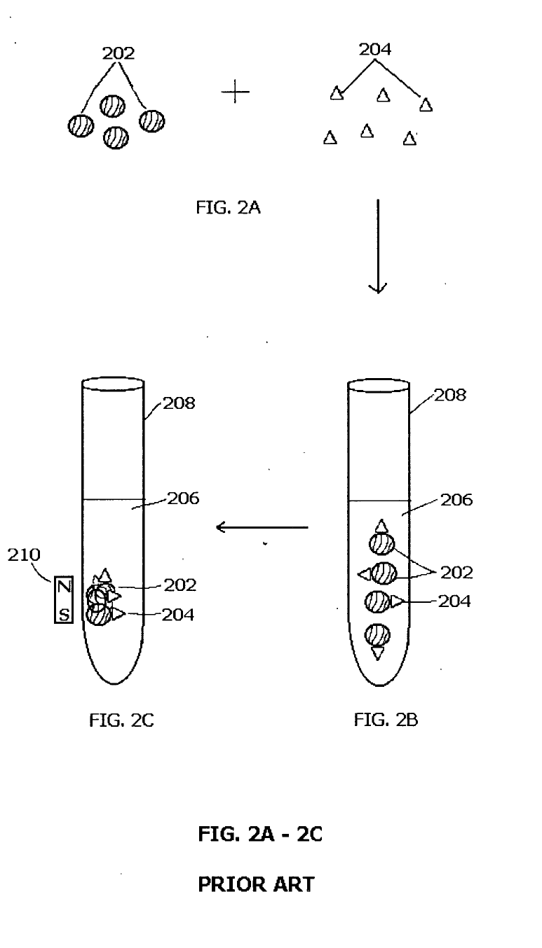

### DETAILED DESCRIPTION OF THE PREFERRED EMBODIMENT

Aspects of the present invention provide systems and methods for producing biochip microarrays. The following description is presented to enable a person skilled in the art to make use the invention. Descriptions of specific applications are provided only as examples. Various modifications, substitutions, and variations of the preferred embodiment may be apparent to those skilled in the art, and the generic principles defined herein may be applied to other embodiments and applications without departing from the spirit and scope of the invention. Thus, the present invention is not intended to be limited to the described or illustrated embodiments, and should be accorded the widest scope consistent with the principles and features disclosed herein. The cited prior art and publications are fully and completely incorporated herein by reference in their entirety.

It will be understood that while numerous preferred embodiments of the present invention are presented herein, numerous of the individual elements and functional aspects of the embodiments are similar. Therefore, it will be understood that structural elements of the numerous apparatus disclosed herein having similar or identical function may have like reference numerals associated therewith.

Embodiments of the present invention are directed toward magnetic array substrates, the biochips resulting therefrom, and methods for fabricating such arrays. Disclosed herein is a simple, uniform platform system for rapidly processing test samples at a high throughput rate, particularly when low sample volumes are present, the platform system being compatible with miniaturized formats to detect and quantify molecules of interest in clinical, pharmaceutical and research environments. The system may also be used for screening chemical compounds, such as organic or organic catalysts, reactants, guests and hosts, etc.

According to embodiments of the present invention, magnetic particles are used to immobilize biomolecular targets in an array format by chemically conjugating the biomolecule to the surface of the magnetic particle, and then arranging the magnetic particles on a substrate comprising a magnetic layer. Compared to flat surfaces, magnetic particles present an increased surface area for immobilization relative to the flat portion of the substrate surface (its so-called projected “footprint”) that a spherical object presents when attached to the substrate. Magnetic particles also may provide greater assay sensitivity and flexibility. Furthermore, by selecting magnetic particles with the desired chemical functionality (by coating the magnetic particle with a polymer, for example), a user can select any conjugation chemistry suitable for the biomolecule under study, independent of the chemistry of the substrate surface.

Magnetic particles that exhibit no magnetic properties in the absence of a magnetic field are used in a variety of affinity binding assay techniques. These techniques rely upon the binding of a biomolecule to the surface of the magnetic particle, to then isolate the particles by applying a magnetic field to the sample volume. Magnetic separations have been used to sort cells, recover antibodies or enzymes from solution, and purify proteins by removing unwanted material from a suspension. Such a technique is an alternative to centrifugation and filtration because the separation is rapid, and because particles can be customized to a large number of assay types. Magnetic particles offer significant advantages because of their ease of handling including speed, rapid kinetics, convenience, low cost and the large surface area available for biomolecular immobilization.

The present invention provides magnetic array substrates and methods for fabricating positional magnetic particle arrays. A critical aspect of the present invention is that the magnetic particles having conjugated biomolecules on their surfaces are attractively coupled to the surface of a magnetic substrate comprising at least one magnetically active layer, such that the biomolecules are immobilized in a spatially defined and physically addressable manner. The magnetic substrate has a plurality of spaced-apart magnetized sites, where the spacing permits individually addressing each site with one or more magnetic particles.

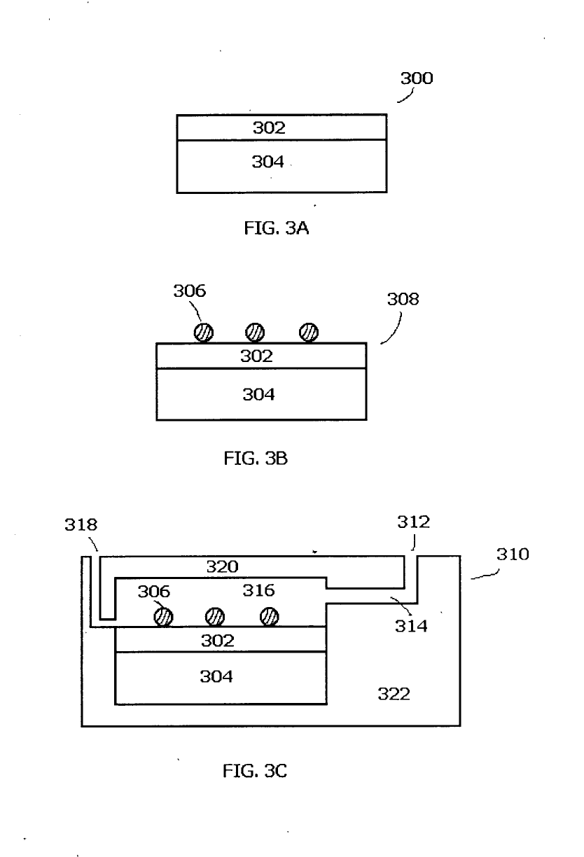

An exemplary embodiment of a magnetic substrate array is illustrated schematically in FIGS. 3A-3C, which is intended to serve as an overview of present invention embodiments before details of each of the system components is presented. Referring to FIG. 3A, an array support layer 302 is disposed adjacent to a magnetic layer 304. The array support layer 302 and magnetic layer 304 comprise a magnetic array support shown generally at 300 in FIG. 3A.

Magnetic layer 304 provides a source of magnetic field to attract magnetic particles 306 toward the magnetic array support 300, as shown in FIG. 3B. Magnetic particles 306 form an array, which in some embodiments may be fixed directly to the magnetic layer 304, but usually the particles (although attracted to the magnetic layer 304) actually contact the array support layer 302. Of course, when an array support layer 302 is used, it is necessary for the magnetic field from the magnetic layer 304 to penetrate the array support layer 302 such that the magnetic particles 306 “feel” the magnetic force from magnetic layer 304, and can respond to that force. The magnetization of the magnet layer may be permanent or created using an electromagnetic source.

The magnetic array support 300 (with or without the array of magnetic particles 306) may be assembled into a holder to construct the complete magnetic biochip shown generally at 310 and FIG. 3C. An important purpose of the holder is that it provides the microfluidic components of the biochip that supply the probe, or chemical reagent, whose binding ability, chemical reactivity, or complementary nature with the immobilize target biomolecules in accordance with the purpose of the assay. Referring to FIG. 3C, the magnetic particle array 308 may be inserted into a holder 322 to form the magnetic array biochip shown generally at 310. The biochip 310 includes a sample fluid entrance port 312, microfluidic capillary channel 314, reaction chamber 316, and a sample fluid exit port 318. A transparent cover 320 may serve as a viewport for observing the assay, which may be necessary, for example, to record the array spots emitting a fluorescent signal during the imaging stage of the process. The sample entrance port 312 is used to provide a fluid containing probe-label complexes, and the sample exit port 318 is used to remove any probe-label complexes that fail to bind to immobilized target biomolecules.

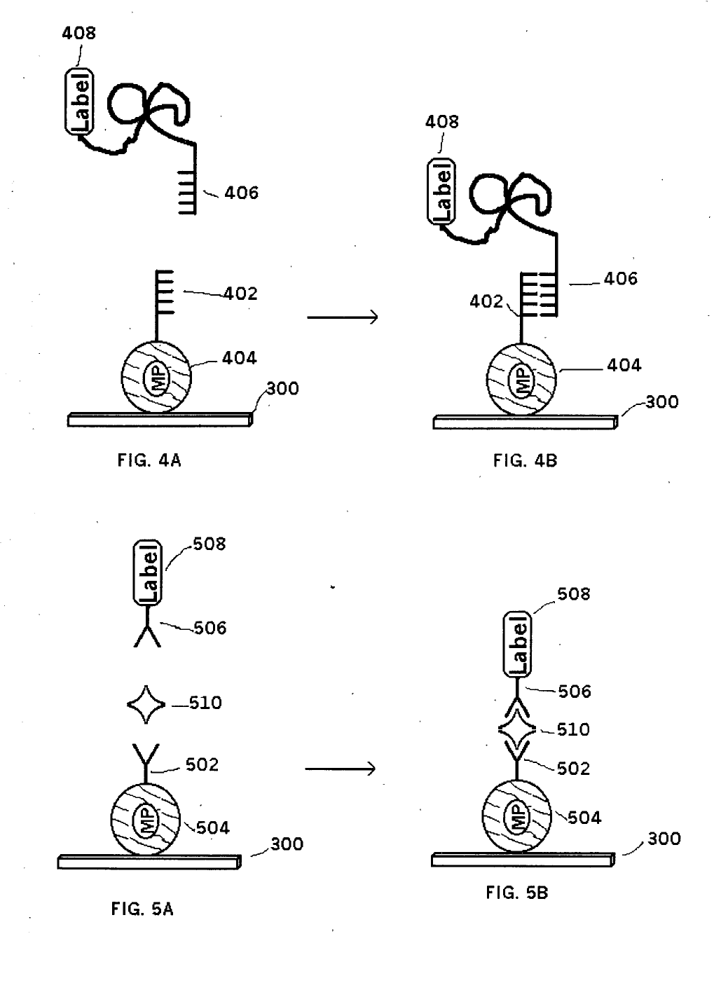

It is useful to review the manner in which biomolecular targets are immobilized on the surface of a magnetic array substrate. Referring to FIG. 4A, a target biomolecule 402 is shown chemically conjugated to a magnetic particle 404, which in turn is magnetically attracted and retained on the magnetic array support 300. Target biomolecule 402 may be, for example, a sequence of messenger RNA (mRNA). A probe 406 has previously been attached to a label 408, which may be a fluorescent marker. Since probe 406 (in this case) is a sequence of DNA complementary to target mRNA 402, probe 406 may hybridize with target 402 such that the array site in which magnetic particle 404 is positioned will now emit a fluorescent signal. Thus, an imaging system detecting the presence of a fluorescent emission at the site occupied by magnetic particle 404 in FIG. 4B relays the information that the target biomolecule 402 is indeed a complementary match to the probe sequence 406. It will be appreciated that the lack of a fluorescent signal from an array site indicates that that particular target biomolecule is not a complementary sequence, and that binding to the probe most likely did not occur.

Similarly, the target biomolecule may be an antibody, as indicated by reference numeral 502 in FIG. 5A. Antibody 502 is chemically conjugated to magnetic particle 504, but it is also bound to an antigen 510. The probe in this immunoassay example is antibody 506, and the assay is probing for array sites having antigen capable of binding to the antibody target 502. As before, a fluorescent label 508 is attached to the antibody probe 506 before the microarray is exposed to the probe. Upon flowing labeled probe 506 through the microfluidic capillary system of the biochip, those targets having a binding capability to probe 506 will capture the probe, and hence that array site will display a fluorescent signal from label 508. An array site with a captured probe 506 is shown in FIG. 5B.

It will be appreciated by those skilled in the art that there is a significant difference in the way biomolecules are immobilized on a substrate surface when comparing embodiments of the present invention and prior art techniques. By using a magnetic particle as an intermediary component, the system displays greater chemical flexibility in the control over which types of targets are immobilized, and greater throughput because the targets may be immobilized to the magnetic particles prior to carrying out the assay (in other words, “off-line”).

Having completed an overview of various embodiments of the present invention, the details of the array support layer, magnetic layer, magnetic substrate array holder, and biochip microfluidic shall now be described. Following that, a discussion will be presented that is directed toward the advantages offered by a magnetic microarray biochip.

Referring again to the FIG. 3A, a magnetic array substrate is generally indicated at 300. The magnetic array substrate 300 comprises an array support layer 302, and a magnetic layer 304. The array support layer 302 may comprise a sheet consisting of glass, plastic, paper or a polymer film having thickness ranging from molecular dimensions to about 0.5 inches. The magnetic layer 304 may be made of a sheet-like material obtained by adding magnet particles to a synthetic resin having at least one surface with N and S poles pattern formed by multiple pole magnetizations. An optional protective sheet or polymer layer (not shown in FIG. 3C) may be formed on the array support layer 302.

Preferred materials of the magnetic array substrate 300 provide physical support for the magnetic particle array 308 and endure the conditions of the deposition process and many subsequent treatments, handling, or processing that may be encountered in the use of the assay in question. The layers of comprising the magnetic array substrate 300 may be fabricated from a variety of different materials, including both flexible and rigid, and/or porous and non-porous materials. By flexible is meant that the array support is capable of being bent, folded, or similarly manipulated without breakage. Examples of array support materials which are flexible solid supports with respect to the present invention include, but are not limited to, membranes, paper, gel pads, flexible plastic films, and the like. By rigid is meant that the support is solid and does not readily bend, i.e. the support is not substantially flexible.

In those embodiments wherein the array support is semi-solid, the semi-solid support preferably comprises an array support layer affixed to a solid and rigid support. Examples of suitable semi-solid array supports for the purpose of the present invention include, but are not limited to, agar, gel pads, agarose, gelatin, polyacrylamide, polyurethane, dextrins, cellulose, polyacrylates, hydrogels and suitable combinations thereof. Suitable semi-solid supports preferably allow biomolecules to diffuse a limited distance into the array support layer 302.

Following spotting or dispensing of the magnetic particle array 308 onto the array support layer 302, the magnetic particles are preferably confined within such a semisolid support to minimize any movement of the immobilized magnetic particles 306, and to provide an optimal surface for the binding reaction.

The magnetic array substrate 300 upon which the magnetic particle array 308 is disposed may take a variety of configurations depending on the intended use of the array (i.e., the type of assay). The shape of the magnetic array substrate 300 in a plan view may be rectangular, square or disc shaped. In many embodiments, an overall rectangular configuration, as found in standard microtiter plates and microscope slides, is preferred. Generally the length of the magnetic array substrates will be at least about 2 mm and may be as long as 600 mm or more, but will usually not exceed about 250 mm and may often not exceed about 200 mm. The width of the magnetic substrate will generally be at least about 2 mm and may be as great as 600 mm or more, but will usually not exceed 250 mm and will often not exceed 200 mm. The height of the magnetic array substrate 300 will generally range from 0.01 mm to 20 mm, depending at least in part on the materials from which the magnetic array substrate is fabricated, and the thickness of the material required to provide the requisite rigidity. In many situations, it will also be preferable to employ materials that are transparent to light, but this is not requirement. For the present invention, the array support layer 302 is fabricated from magnetically permeable materials.

The array support layer 302 may be fabricated from a variety of materials. For instance, the array support layer 302 may be glass, Si, Ge, GaAs, GaP, SiO₂, Si₃N₄, modified, or any one of a wide variety of gels or polymers. Exemplary polymers include polytetrafluoroethylene, polyvinylidenedifluoride, polystyrene, polycarbonate, polypropylene, and combinations thereof. Other exemplary materials include, acrylates, styrene-methyl methacrylate copolymers, ethylene/acrylic acid, acrylonitrile-butadienestyrene (ABS), composites such as ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polycarylonitrile (PAN), polyacrylate, polycarbonate, polybutylene terephthalate (PBT), polyethylene terephthalate (PET), polyethylene (including low density, linear low density, high density, cross-linked and ultra-high molecular weight grades), polypropylene homopolymer, polypropylene copolymers, polystyrene (including general purpose and high impact grades), polytetrafluoroethylene (PTFE), fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoroethylene (ECTFE), polyvinyl alcohol (PVA), silicon styreneacrylonitrile (SAN), styrene maleic anhydride (SMA), thermoplastic polyurethanes, polyesters such as polyethylene terephthalate, nylon polymers such as nylon-11, nylon 12, block polymers of polyethers and polyester, natural rubber, polyamides, polyolefins such as polyethylene, polypropylene, synthetic rubbers, thermoplastic hydrocarbon elastomer, nylon and polypropylene) paper, cellulose and blends thereof, and the like; metals, e.g. gold, platinum, coated steel, magnet composition, metal oxides, and glass. In a preferred embodiment the substrate is glass or plastic. Surfaces on the array support substrate usually, though not always, are composed of the same material as the substrate. Accordingly some preferred array support or substrate material may include, but not limited to, silica based substrates such as glass, quartz, silicon, polysilicon, with or without insulating coating layers such as silicon oxide, layers of polymeric materials e.g. plastics such as polymethylmethacrylate (PMMA), polycarbonate, polytetrafluoroethylene (TEFLON), polyvinyl chloride (PVC), polydimethylsiloxane (PDMS), polysulfone, polystyrene, polymethylpentene, polypropylene, polyethylene, polyvinylidine fluoride, ABS (acrylonitrile-butadiene-styrene copolymer), and the like. These polymeric materials are preferred for their ease of manufacture, readily availability, low cost and disposability as well their general inertness to extreme reaction conditions. Again, there polymeric materials may include treated, derivatized, coated or modified surfaces to enhance their utility, e.g. coating with hydrophobic polymer to confine the liquid movement or provide enhanced fluid direction.

Thus, the array support layer 302 may be composed of any of a wide variety of materials, for example, polymers, plastics, resins, polysaccharides, silica or silica-based materials, carbon, metals, inorganic glasses, membranes, or any of the above-listed substrate materials or combinations thereof.

Additionally, the array support layer 302 may be treated, coated, modified, printed or derivatized using polymers, chemicals to impart desired properties or functionalities to the array support surface. The array support layer materials are also generally selected for their compatibility with full range of conditions to which the array substrate may be exposed, including extremes of pH, temperature, salt concentrations, solvents, and application of electric fields. The array substrate layer materials are selected for their thermal, optical, surface properties as well as their compatibility with manufacturing techniques (lamination, injection molding, stamping, embossing and other techniques).

The array support layer 302 may be smooth, having a substantially planer surface, or it may contain a variety of structures such as wells, grooves, multi-well plates, dishes, screens or fine mesh, depressions, elevations, trenches, chambers, nanowells, channels, gel pads, and the like. There may be microfluidic devices associated with the array support layer 302 (these devices may be formed within the layer), such as dipstick, strip, tube, cuvette, capillary, flow-thru devices or screens. Capillary devices fabricated within the array support layer 302 may be fabricated by known microfabrication techniques, such as wet chemical etching, photolithographic techniques, controlled vapor deposition techniques, and laser drilling.

The surface of the magnetic array substrate 300 may be modified, treated, printed, coated with one or more different layers of patterns of compounds that serve to modify the properties of the surface in a desirable manner. In addition, where surface modification is contemplated, the magnetic array substrate 300 should be chemically treatable to enhance the array or assay performance.

According to certain embodiments of the present invention, the surface of the magnetic array substrate 300 may suitably treated, coated, printed, derivatized, layered or modified with one or more different layers of compounds that serve to modulate the properties of the surface in a desirable manner. Such treated or modification layers, when present, will generally range in thickness from a monomolecular thickness to about 5 mm, and may include combination of layers. Modification layers of interest include, but not limited to, inorganic and organic layers such as metal, metal oxide, polymers, small organic molecules, hydrogels, sugars, electrically conductive or insulating layers and the like. Polymeric layers of interests include layers of proteins, peptides, polynucleic acids and mimetics thereof, e.g., peptide nucleic acids and the like, polysaccharides, hydrophobic or hydrophilic polymers, polycarbonates, polyesters, polyurethanes, polyacrylamides, polyureas, polyamides, polyolefins, polysiloxanes and the like, where the polymer may be hetero or homopolymeric, and may or may not have functional moieties attached thereto, e.g. conjugated. Thin film forming water soluble polymers include alkyl and hydroxy alkyl celluloses, Hyaluronic acid, sodium chondroitin sulfate, Polyacrylic acid, polyacrylamide, Polycyanoacrylates, Cyclodextrin, polydextrose, dextran, gelatin, polyvinyl alcohol, polyvinyl pyrrolidine, polyalkylene glycols, polyethylene oxide, carboxymethylcellulose, chitosan, alginates, polydextrin, collagen, maltodextrin, natural gums, agar, carrageenan, polyethylene glycol, polymers of methyl methacrylate, polydextrose, pectin, starch, microcrystalline cellulose and the like.

The treated surface may facilitate the immobilization of the magnetic particles 306 by the magnetic array substrate 300, and minimize any non-specific binding to the surface of the magnetic array substrate 300 and improve the detection of signals from the magnetic particles, and improve stability of biomolecules.

Such treatment also preferably facilitates reagent confinement to the desired array positions. Suitable treatment may include the entire surface of the magnetic array substrate 300 array support, or just specific locations on the array substrate, such as regions surrounding each of the array spots. In certain embodiments, certain of the aforementioned regions are left untreated. Generally, in performing the methods of the present invention, positions of the printed array locations and the areas surrounding those locations receive different treatments (or no treatments), coatings, derivatizations, or modifications. The differential treatment facilitates confinement of individual species of immobilized test molecules to their respective array receptacles.

The treatment, coating or derivatizations of the array support is generally performed prior to the spotting of the magnetic particles onto the magnetic array substrate 300. However, it is contemplated that the treatment, coatings, or derivatizations is performed contemporaneous or subsequent to the addition or immobilization of the biomolecules and or magnetic particles onto the array support. UV cross-linking is an example of a treatment generally performed subsequent to the addition of the biomolecules or magnetic particles to the array support. Another example is the polymer coating of the array surface for improving the stability of the array, which may be performed subsequent to the immobilization of magnetic particles and biomolecules onto the array substrate. Low salt immobilization of nucleic acid is an example of a treatment generally performed contemporaneous with the addition of the molecule to the array substrate.

Treatments, coatings, or derivatizations suitable to the methods of the present invention include, but are not restricted to poly-L-Lys, streptavidin, cellulose, hydrogel polymerization, antibodies, cells, dextrins, polypeptides, silane derivatives, low salt, plasma, photo-irradiation, or acid. Choice of treatment, coating or derivatization is preferably guided by the nature of the array support and the molecules that are to be immobilized in an array. A variety of suitable attachment chemistries are well known in the art.

According to those embodiments wherein the reagent solution is polar, the surface of the magnetic array substrate 300 at the positions of the array spots are preferably hydrophilic or treated so as to be hydrophilic. To facilitate reagent confinement in such embodiments, the area surrounding each array spots is preferably hydrophobic or treated so as to be hydrophobic. Conversely, in those embodiments wherein the reagent solution is relatively non-polar, the positions of the array spots are preferably hydrophobic or treated so as to be hydrophobic. Similarly, in such embodiments, the area surrounding each array spots is preferably hydrophilic or treated so as to be hydrophilic to facilitate reagent confinement. In certain embodiments of the present invention, however, both the positions of the array spots and the area surrounding the array spots are treated so as to both be hydrophobic or both be hydrophobic.

According to the embodiments of the present invention, methods for treating or modifying the surface of the magnetic array substrate 300 include laminating, printing, layering, or coating, according to any one of the methods known in the art. One example of this would be creating array receptacles through the use of a suitable stamp or stamp pad such as self-inking soft porous plastic stamp pad.

Alternatively, a stamp pad with desired pattern can made and used for specific treatment, coatings, and derivatizations of the magnetic array substrate 300. Other layering techniques may include ink jet printing, offset press printing, serigraph printing, silk screening, lithography, flexography, intaligo, thermal laser printing, and Heidelberg printing. In addition, screens or masks comprising various structures such as channels, wells, chambers, circles, squares, rectangles, grooves and ridges structures can be applied or laminated to the substrate surface and coated with an additional materials.

Some of the array supports that can be used in the invention are readily available from commercial suppliers. In a one embodiment, the array support is a 96˜, 384˜, or 1536˜ or more—well microtiter plates, glass slides sold by Corning Costar and Erie Scientific and others. Alternatively array support comprising surface comprising different geometries such as dimples, or indentations can be formed by micromachining on a substance such as aluminum or steel to prepare a mold, then microinjecting plastic or similar material into the molds to form such structures.

Next, the magnetic layer 304 will be described, particularly in the context to its role as part of the magnetic array substrate 300. In preferred embodiments, the magnetic layer 304 (in association with the array support layer 302, and as a critical component of the magnetic array substrate 300) generates the magnetic field intensity that attracts the magnetic particles 306 to the surface of the magnetic array substrate 300, and causes them to form the magnetic particle array 308. In this manner, the biomolecules 402/502 are immobilized on the surface of the magnetic array substrate 300.

The terms magnetic layer, magnetic sheet, and magnetic film are used herein to mean at least one substantially flat layer, sheet, or thin film having a magnetic field of sufficient strength to attract the magnetic particles 306, and to securedly hold those particles in their respective positions of the array. If a array support layer 302 has been disposed on the surface of the magnetic layer 304, then it will be obvious to those skilled in the art that the magnetic field from the magnetic layer 304 will be able to penetrate the support layer 302 such that the magnetic particles 306 and the array 308 will be exposed to the field.

The magnet layer will have individual discontinuous small sites of enhanced magnetization separated by a continuous region of lower magnetization, usually non-magnetic. The sites will generally be in evenly spaced rows and columns, although any spatial organization may be employed for specific applications. The spacing between particles is desirably related to the spacing of vessels which serve as the source of the magnetic particles, particularly microtiter plates that today range from 12 to 1536. Since the magnetized sites can have a much greater density than the spacing between the well centers, the spacing between magnetized sites will be much smaller than the spacing between the centers of the wells. Therefore there may be 1 or more, even 100 or more, magnetized sites within the spacing of the wells When transferring the magnetic particles from the vessels using the battery of transfer devices, pattern of the vessels will be recreated on the magnetic substrate. One then moves the battery of transfer devices to apply the magnetic particles beginning with the next successive magnetic site in a row or column, so as to repeat the positioning of the magnetic particles in relation to the vessel spatial arrangement. This can be repeated until all of the magnetic sites are occupied. In this way one can obtain magnetic particles from a plurality of microtiter well plates or repetitively transfer magnetic particles from the same plate or combination thereof. Because the magnetic sites maybe of minute size a very dense distribution of magnetic sites can be achieved, while still avoiding interference between sites.

The magnetic layer 304 may have the mechanical properties of being either rigid or flexible, and the optical properties of being either opaque, transparent, or translucent depending on its composition.

An exemplary magnetic layer 304 includes, but is not limited to, a film consisting of a fine magnetic powder such as barium ferrite loaded into a thermoplastic binder, strontium ferrite based materials, bonded materials comprising neodymium, iron, and boron, a sheet of plastic or vinyl material impregnated with a ferromagnetic material, a sheet of synthetic resin material having mixed therein a magnetic powder magnet particles embedded in a polymer sheet of 0.001 inch to 0.5 inches thickness, a vinyl material including magnetic materials dispersed therethrough or other suitable material having properties compatible with its intended purpose.

As apparent to those skilled in the art, the thickness of the magnetic layer 304 will vary depending on factors which include the composition of the layer material, number of magnetic sheets comprising the layer, the desired field strength, the spacing and number of the magnetic poles, the type of magnet particles 306 used, and the manufacturing process used to fabricate the magnetic layer 304. In general, the thickness of the magnetic layer 304 may range from about 0.001 inches to about 0.5 inches.

Exemplary magnetic layers have a magnetic field strength in a range, which includes, but is not limited to, about 150 to about 10,000 Gauss. It will be appreciated by those skilled in the art that this magnetic field strength is significantly lower than magnetic field strengths used in conventional magnetic separation techniques, and this may have advantages in terms of potential damage to biomolecules that are susceptible to large magnetic field strengths. For permanent magnet sheets or films, the magnetic field strength of the external magnetic means at the pole faces should be in a range of about 100 to 10,000 Gauss, and preferably greater than about 400 Gauss.

The preferred distance between the magnetic layer 304 and the array support layer 302 or container device is generally less than 0.500 inches. The field strength of the magnetic layer 304 should be large enough, and the distance between the magnetic layer and the array support layer short enough to give an efficient and stable capturing of the magnetic particles. Alternatively, electromagnets with suitable field strengths can be used in embodiments of the present invention, so that a value of the magnetic field strength of the magnetic layer makes sense when the electromagnet is turned on.

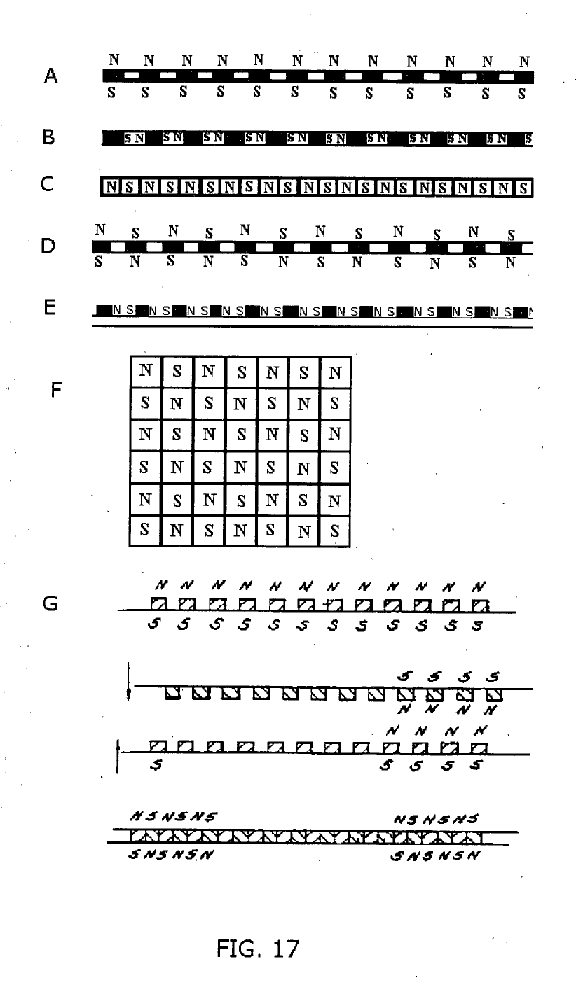

The magnetic layer 304 may be formed by extrusion molding of a synthetic resin material containing magnet particles into a sheet. It has N and S poles formed on one of the surfaces by multiple pole magnetizations, alternately arranged at a constant pitch of 0.1 to 5 mm. FIG. 17 illustrates the examples of magnetic multi-pole patterns for the magnetic layer 304. The thickness of magnetic layer 304 is suitably selected based on the magnetic strength required to attract, immobilize and hold magnetic particles, and there are other considerations such as manufacturability, practicability of handling, and the resistance to conditions normally encountered in conducting binding assays.

Methods of manufacturing the magnetic layer 304 include lithographically, vapor deposition, calendering, extrusion, injection molding, and electroplating. In another embodiment, the magnetic layer 304 can also be deposited on a solid support by means of electron beam evaporation, sputtering or other deposition techniques well known to those of skill in the art.

Permanent magnets for practicing the present invention preferably are thin sheets or thin film form and have a surface field strength sufficient to attract the magnetic particles and hold them throughout the microarray processing steps. Permanent magnets of rare earth alloys having a surface field strength in the range of lower hundred Gauss to several kilo-Gauss are preferred. Exemplary materials from which a magnetic layer 304 may be made from permanent magnetic materials include neodymium-iron-boron or samarium-cobalt magnets, characterized by a BHₘₐₓ (maximum energy product) in the range of 0.5 to 15 MGOe (megaGauss Oersted). A sheet magnet is preferably oriented with its magnetic lines of force perpendicular to the vertical axis of the substrate. Alternate cross-sectional shapes, orientations, and magnetic pole orientations with respect to the array substrate or device or container are also embodiments of the present invention. Examples of some of the multipole magnetic patterns include but not limited to those shown schematically in FIG. 17.

Generally the magnetic layer 304 is placed or mated in close proximity to the array support layer 302. The distance between magnet layer and the container is between about few microns to about 10 mm to create a desired magnetic field strength within the magnetic field cavity of the test medium.

The field strength created in the magnet field cavities should be carefully balanced so that it is sufficient to pull the particles in a timely fashion, and immobilize the particles on the array substrate. In certain situations involving the processing of a plurality of biochip containers, it may be advantageous to employ a magnetic layer with a high field strength since the magnetic layer will then be sandwiched between several layers of array support layers.

If the magnet is a permanent magnet, it preferably comprises a rare earth composite type such as neodymium-iron-boron or samarium-cobalt and has a surface field strength of about 200 Gauss to 15 kiloGauss, which is sufficient to attract the magnetic particles in the size range of about 0.2 micron to 1000 micron. Though in one embodiment of the present invention the magnetic layer 304 comprises a permanent magnetic material, an electromagnetic device may also be employed in place of the permanent magnet.

In exemplary embodiments of the present invention, typical magnetic properties of the magnetic layer 304 follow:

1) magnet layer thickness from 0.0001 to 0.500 inches

2) magnet energy products from 0.5 MgOe to 15 MgOe

3) magnet Br ranging from 100 g to 10,000 g

4) magnet Hc's from 1270 Oe to 3930 Oe

5) different multiple pole spacings from 0.002 to 0.500″

6) Magnetic field strength (magnetic flux) from 0.1 KGauss to 15 KGauss.

The magnetic layer 304 has now been described, particularly in relation to its role as part of each magnetic array substrate 300. Next, a discussion will be given regarding the bonding of the array support layer 302 to the magnetic layer 304, ultimately to describe the structure of the magnetic array support 300.

According to embodiments of the present invention, the array support layer 302 and the magnet layer 304 may be mated, bonded, or clamped to one another to form the magnetic array substrate 300. Alternatively, the array support layer 302 and the magnet layer 304 may be positioned adjacent to one another by any number of means that do not involve bonding, such as by sliding the array support layer 302 into the bottom slot of a holder, and the magnetic layer 304 into the upper slot of the holder.

Attachment of the array substrate and magnet layer of the magnetic array substrate is typically accomplished by well known methods, including adhesive bonding, acoustic or ultrasonic welding, RF welding, solvent welding (particularly for polymeric layers), anodic bonding, thermal bonding (including thermo-melting bonding), and the like. The thickness of bonding region between the two layers is very small compared to the overall thickness of the magnetic array substrate 300.

When the two layers are bonded together, bonding is generally carried out under any number of methods or conditions known in the art, which may vary depending upon the nature of the substrate materials used. For example, the two layers may be thermal bonded, particularly in the case of substrates that include glass, silica based materials, and polymer based substrates. Thermal bonding typically comprises mating the two substrates that are to be bonded under conditions of elevated temperature. In some cases, the application of external pressure may be required. Generally, the range of temperatures and pressures used in the bonding procedure will vary depending upon the nature of the substrate materials.

When polymeric substrates are involved in the thermally bonding process (such as a polymeric array support layer 302, or a polymeric magnetic layer 302 having magnetic species impregnated therein), the bonding process will typically utilize lower temperatures and/or pressures than silica-based substrates in order to prevent any thermal degradation, distortion, or damage in other ways to the substrates layers. In general, temperatures for bonding a polymer layer to another layer (which may also be a polymer layer) will vary from about 80 to 200 degrees Celsius, including any range subsumed therein. Because of the reduced temperatures required for bonding polymeric substrates, such bonding may typically be carried out without the need for high temperature ovens, which are generally required for the bonding of silica-based substrates.

Adhesives may also be used to bond the array support layer 302 to the magnetic layer 304. In accordance with well known methods, a layer of adhesive is disposed between the two layers to be bonded, and optionally, the two layers may be pressed together (in other words, pressure is applied to the composite) until the adhesive “sets.” A variety of adhesive types may be used in accordance with these methods, including, for example UV-curable adhesives hot melt adhesives, and epoxy adhesives. In particularly preferred aspects, the selected adhesive is electrically insulating, i.e., nonconductive, non-soluble and/or non-leaching in application buffers. The adhesive preferably has other properties, such as being low in fluorescent activity, so that any optical emissions from the bonding or adhesive portion(s) of the magnetic array substrate 300 will not interfere with fluorescent signals emitted from a probe-label complex. The preferred method of bonding an array support layer 302 to a magnetic layer 304 according to embodiments of the present invention involves commercially available adhesive formulations, or tape, that may be obtained from, for example, 3M Corporation, Adhesive Research Inc., Martek Corporation, and Master Bond Inc.

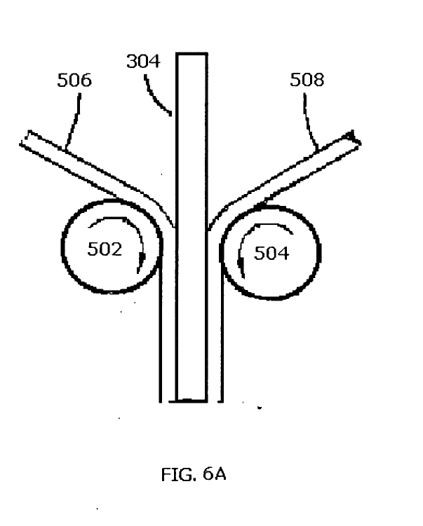

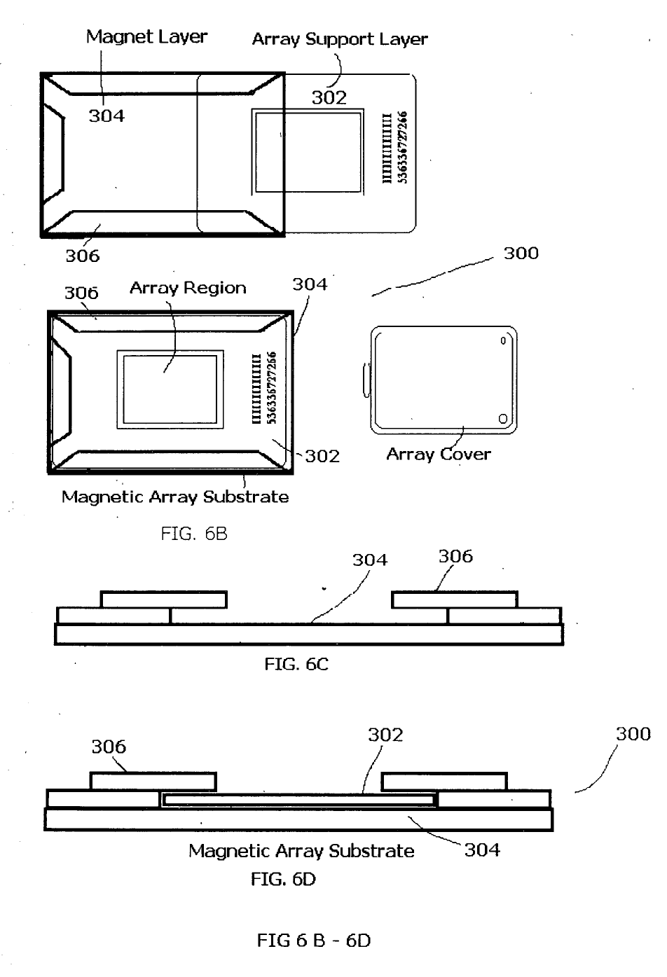

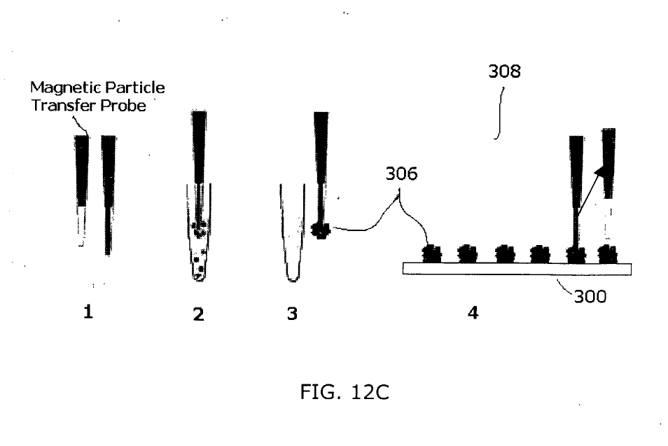

In alternate embodiments, the array support layer 302 can be attached to the magnetic layer 304 by simple fastening mechanisms such as screw clamps, clip-style clamps, brackets, hinged devices with tightening means, and the like, as shown in FIG. 6B. These clamping devices clamp the edges of the array support structure and magnet layer. In such instances, the array support layer 302 is compressively clamped against the magnetic layer 304 to form a sealed, joined structure. Also shown in FIG. 12B is the array cover which may be used during the microarray experiment to minimize evaporation of reagents and for the protection of magnetic particle array during storage. Alternatively, integrated clamping mechanisms are provided as a portion of the magnetic layer, into which the array support layer is snapped, as shown in FIG. 6C-D.

In another method of bonding that layers of the magnetic array substrate 300 together, shown in FIG. 6A, the magnetic layer 304 is passed through a pair of opposed rollers 502 and 504. Referring to FIG. 6A, a first polymer sheet 506 extends through the rollers 502, 504 between the magnetic layer 304 and the roller 502. A second polymer sheet 508 extends through the rollers 502, 504 between the magnetic layer 304 and the roller 504. Thus, according to this embodiment, the magnetic layer 304 is sandwiched between the polymer sheets 506 and 508, producing a product similar to the magnetic array substrate 300 shown in FIG. 3A. The first polymer sheet 506 might be for example, the array support layer 302, and the second polymer sheet 508 may be, for example a protective coating (not shown in FIG. 3A) on the opposite side of the magnetic layer 304 (which may be a non-magnetized surface of the magnetic layer).

The polymer sheets 506, 508 preferably each have a thickness ranging from monomolecular thickness to 400 mils. The magnetic particles 306 are attracted strongly to the magnetic sheet 304 (having a thickness of about 5 mils) even when polymer layers are laminated to it having thickness up to 400 mils (0.400 inch) thick.

Alternative methods of forming the magnetic array substrate 300 are contemplated. For example, a liquid polymer may be applied to the magnetic layer 304, and then subsequently cooled or cured to form a polymer layer laminated to the magnet layer. Similarly, a liquid polymer could be spray coated onto the magnet layer. In alternate embodiments, vapor deposition, extrusion, casting and roll laminating are also contemplated.

In addition to laminating polymers of only one type to a side of the magnetic layer, a composite of preformed multi-layer laminates could be coated on one or both sides of the magnetic layer 304. Such a laminate could be a fusible polymer, polyester, acrylate, polycarbonate and paper laminate. These multi-layer laminates may be mounted to both sides of the magnetic layer 304, or only one side. Of course, it is possible to laminate the composite to one side of the magnetic layer, and a layer comprising only one type of polymer to the other side.

Magnetic array substrates of the present invention may be incorporated into a biochip structure that provides ease of analysis, high throughput, and the potential for an inexpensive disposable system. Furthermore, the structures in which the magnetic array substrates are suitable in a variety of formats, including biochip formats, a multi-well formats, capillary formats and the like. The devices typically are designated on a scale suitable to analyze very small volumes of sample, often as small as 500 microliters or less. Analytes present in very low concentrations (e.g., Picogram or nanogram quantities) in such small volumes of sample fluid can be rapidly analyzed, often in as little as 30 minutes or less.

Biochips with magnetically immobilized biomolecules will typically comprise a holder with a nesting site for positioning the chip in relation to the microfluidic transport system. Biological sample fluids such as blood, plasma, serum, urine, sputum, and saliva and like suspected to contain a particular analyte, cellular contaminant, or toxin is deliver to the microfluidic transport portions of the biochip via an inlet port. Fluids may travel through the microfluidic channels or chambers actively as a result of an active pumping force, or passively as a result of capillary action.

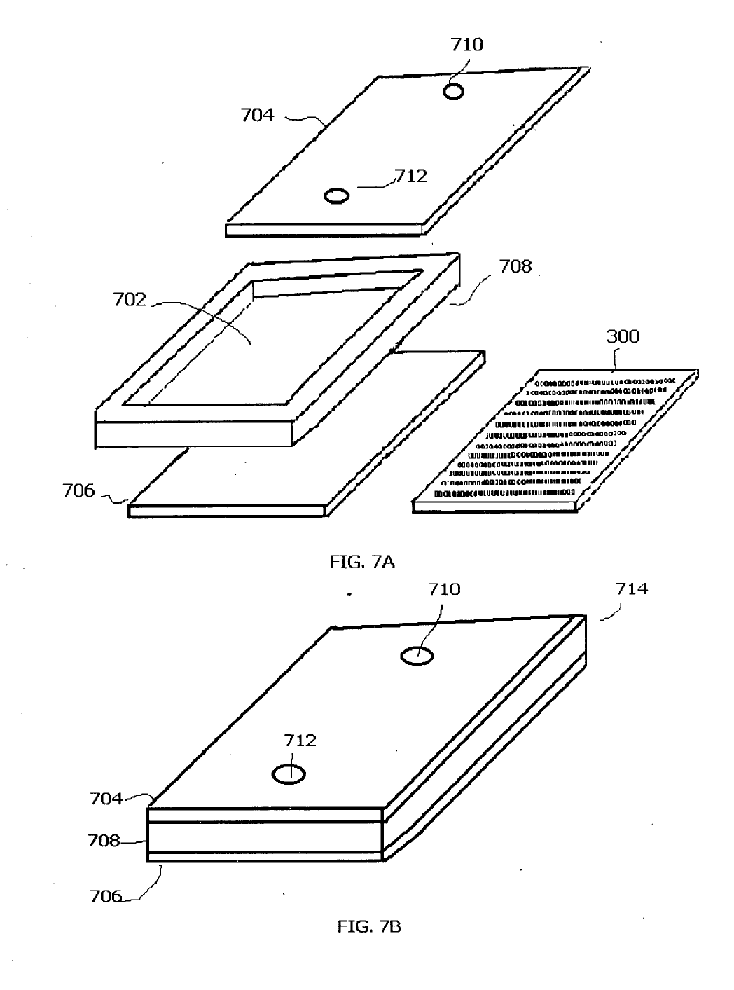

In its simplest embodiment, the magnetic array substrate 300 may be inserted into a biochip device comprising a substantially rectangular shaped cartridge, or holder, for housing the magnetic array substrate 300, the cartridge having a fluid entry port and fluid exit port. This biochip device is illustrated in a disassembled form in FIGS. 7A, where a reaction cavity 702 is a formed by a space bounded on the top and bottom by substantially rectangular plates 704 and 706, respectively, and on the sides by a sidewall spacer 708. The magnetic array substrate 300 is inserted into the reaction cavity 702, and may be supported by either of the plates 704, 706, or by the sidewall spacer 708, or by combinations thereof. Any of the pieces forming the reaction cavity 702 may contain ports for introducing assay reagents to the reaction cavity 708 (and thus the magnetic array substrate 300), and for exiting waste products after the assay has been completed. In the exemplary biochip illustrated in FIG. 7A, the top plate 704 has an inlet port 710 and an exit port 712. An assembled biochip device 714 is illustrated in FIG. 7B.

The term container, cartridge, reaction vessel, or reaction chamber is used to mean a chamber or capillary device for holding a fluid, where the chamber has at least one wall having one or more inner surfaces and one or more outer surfaces; and at least one aperture comprising an inlet to allow for the introduction of one or more substances/liquids into the device; an outlet for the withdrawal or removal of one or more substances from the container. The chamber may also have a venting port for releasing gases from the chamber as sample is introduced into the chamber. Each of the apertures (or ports) may be closable or sealable to prevent the contents of the device from leaking out, and to prevent the evaporation of liquids. Such containers are often referred to as hybridization chambers, binding reaction devices, lateral flow devices, or capillary or microfluidic devices.

In some embodiments, the channels in the device that conduct fluid from the sample inlet port to the reaction chamber, and from the reaction chamber to the waste exit port, are capillary tubes. In other words, test sample fluid movement through the device relies on capillary forces. Additional chambers and capillaries may be added to customize a device, or to tailor the device to serve a specific function. For example, one or more capillaries may be used to transport the test sample from the inlet port to the reaction chamber. Likewise, one or more capillaries may be used to remove fluid from the reaction chamber. The flow of fluid through the device does not have to rely on capillary action, however, and a differential pressure may be used to drive fluid flow in lieu of or in addition to capillary forces.

The design of the capillary channels, reaction chamber(s), and microarray(s), including interconnections and flow patterns between structures, and overall dimensions are important in facilitating contact between the immobilized biomolecules in each of the array spots and the fluid molecules of the assay reagents that are being flowed through the biochip. The device comprises a solid substrate, typically on the order of a few millimeters thick, and approximately 0.1 to 5.0 centimeters square, or any other shape fabricated to define a sample inlet port and a capillary flow system. The devices may be as large as 12 by 12 inches. More often, a biochip device may be slightly larger than a conventional glass microscope slide.

The term microcapillary is used herein to define chambers and flow passages having cross-sectional dimensions on the order of 50 micrometers (μm) to about 50 millimeters (mm). Typically, the depth of the capillary flow channels and fluid handling regions range from about 50 μm to about 8 mm. The average width of the channels typically range from about 200 μm to about 8 mm. With regard to the array design, different regions of the microarray may be arranged in both series and parallel configurations, with the different regions connected to one another by capillary flow channels. Channels may also be used to connect other types of adjacent structures on the biochip through which fluid can flow. For many applications, channels widths will vary depending on chip design, sample volume requirements and other parameters, such as the purpose of the assay. Chambers in the device often will have larger dimensions, i.e., on the order of 1 to 500 millimeters or more.

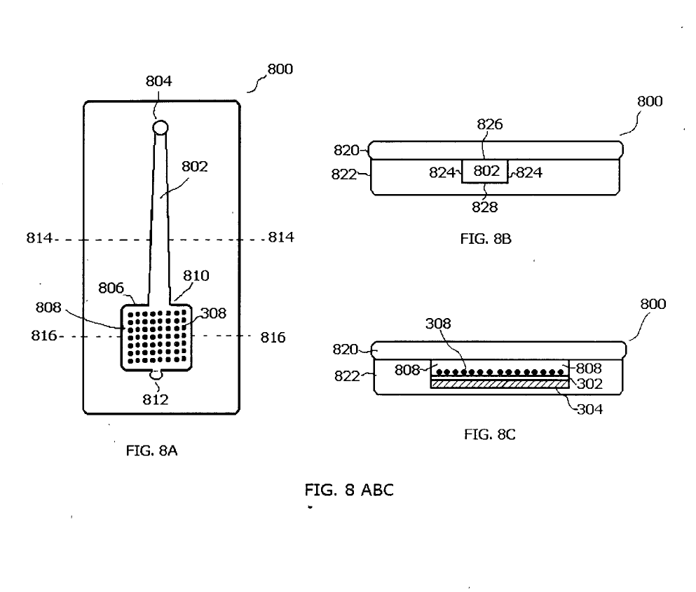

The cross-sectional shape of the microfluidic channels need not be constant along the length of the channels, has illustrated in FIGS. 8A-8C. The biochip device generally indicated that 800 in FIG. 8A has a microfluidic capillary channel with a proximal region 802 into which a sample fluid is transported after being introduced through sample entry port (may also called a fluid addition port) 804. Working toward the magnetic particle array 308 from the channel's proximal region 802 is a distal region 806 of the microfluidic portion of biochip 800. The distal region 806 includes a reaction cavity 808. The proximal region of the channel 802 fluidly connects with the distal region 806 at a junction region 810. It is into this reaction cavity 808 that the magnetic array substrate 300 is inserted to perform the assay. As in previous embodiments, the magnetic array substrate 300 comprises a magnetic layer 304 having at least one magnetized surface, and an array support layer 302 positioned adjacent to the magnetized surface of the magnetic layer. Fluids are removed from the reaction cavity 808 through a fluid exit port 812. The reaction cavity 808 may also contain a vent port 812 to allow gas to escape from the reaction cavity 808, thus facilitating fluid flow through the reaction cavity 808 from the junction region 810 to the exit port 812.

FIG. 8B illustrates a cross-section of the biochip device 800 generally taken along the line indicated at 814 in FIG. 8A. Referring to FIG. 8B, the biochip device 800 further comprises a lid 820 and base 822, which serve to define the cross-sectional shape of the proximal region 802 of a microcapillary of biochip 800. In this embodiment, the distance between lateral walls 824 is appreciably greater than the distance between the bottom surface 826 of the lid 820, and the bottom of the channel 828; this configuration permits fluid flow through the device to be readily viewed by an individual conducting the assay by looking through either a lid window (not shown) positioned over proximal region 802, or by utilizing a transparent or translucent lid 820. Referring again to FIG. 8B, it will be understood that the surfaces creating the greatest amount of capillary force in the proximal region 802 are top surface 826 and bottom surface is 828, respectively.

The channels and chambers in cross-section taken through the thickness of the chip may be triangular, truncated conical, square, rectangular, circular, oval, or virtually any shape.

A cross-section of the biochip array device taken along the lines 816 in FIG. 8A is shown in FIG. 8C. This cross-section is taken through the reaction cavity 808, where a magnetic particle array 308 has been disposed upon an array support layer 302, which in turn is positioned adjacent to a magnetized surface of magnetic layer 304. The immobilized biomolecules on the surfaces of the magnetic particles comprising the magnetic particle array 308 are positioned such that they are exposed to assay reagents flowing through the reaction cavity 808.

The microcapillary flow system may be designed and fabricated from glass or plastic, quartz, polymers, metals, or virtually any sort of solid materials. Conventionally established fabrication methods may use established fabrication methods, or by molding polymeric materials. The capillary flow systems may be used, such as molded polymeric materials in the case of polymers. Biochip devices may be constructed by machining the flow channel(s) and the detection window region(s) directly into surfaces of the substrate. In the case of the detection window, a cover may be positioned over the window and adhered to the substrate, where the cover may be a transparent glass cover or a plastic sheet.

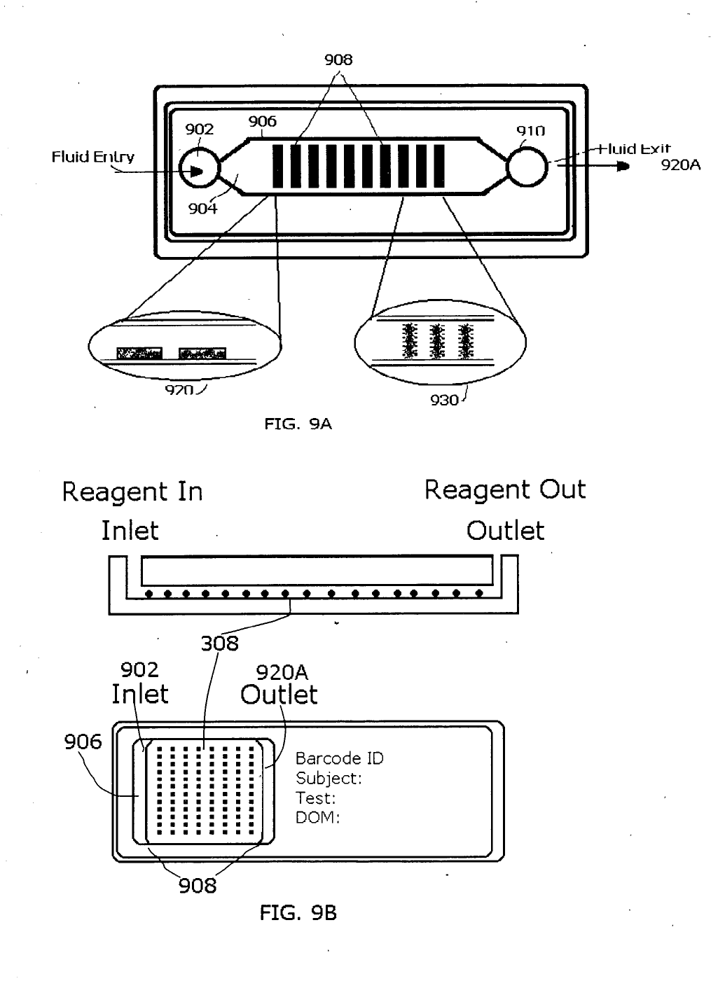

In an alternate embodiment, the microfluidic channel that delivers sample to the microarray assumes a width that is comparable to that of the reaction chamber immediately after leaving the sample inlet port. Referring to FIG. 9, a sample inlet port 902 functions the same way as in earlier embodiments. A microfluidic channel 906 widens almost immediately within a transition region 904 to substantially the full width as that of the reaction chamber 908. In this embodiment, a symmetrical arrangement exists at the exit end of the microfluidic transport channel, and the channel 906 narrows just as it approaches the exit port 910. The magnetic particle array 308 is shown in cross section at 920 as it would appear along the lines 920A, and in more detail at 930, as it would in the same plan view as FIG. 9.

In an alternate embodiment, the microarray spots are printed onto an array support layer 302 of a biochip device wherein each array spot is bounded by raised walls in a manner sufficient to form a plurality of microcontainers or wells. In such a case, each array spot would sit at the bottom of one of the wells. Such high throughput devices are described in U.S. Pat. Nos. 6,242,246 and 6,232,066. By plurality is meant at least 2, usually at least 6 and often at least 24. The number of wells may be as high as 96, and will usually not exceed 100. The volume of each reaction chamber may be as small as 2 microliters (μl), but will usually not exceed 1000 microliters.

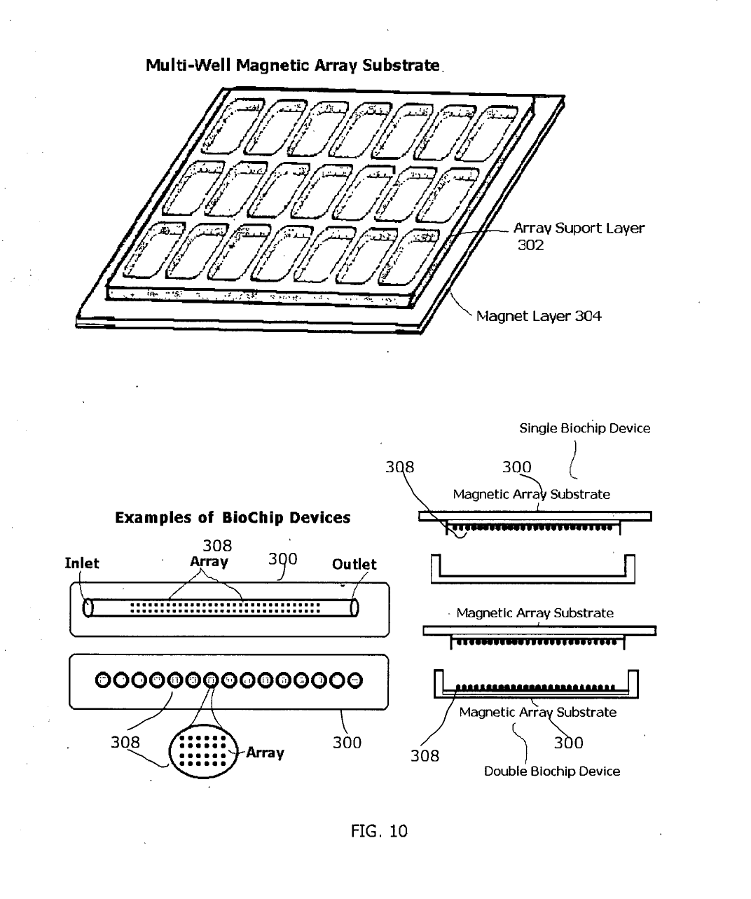

FIG. 10 illustrates exemplary commercial embodiments of the magnetic array substrate and the magnetic biochips of the present invention.

The term “magnetic particles” is to be understood as encompassing so-called magnetic beads, magnetic microbeads, paramagnetic particles, magnetically attractable particles, magnetic spheres, microclusters, and magnetically responsive particles. These terms are frequently found in the literature, and it is to be understood that they are interchangeable. As used herein, “magnetic particles” includes particles capable of being dispersed or suspended in a liquid media without significant aggregation following the application of a magnetic field.

Magnetic particles 306 are formed into an array (using techniques to be discussed shortly) by responding to the attractive forces of a magnetic field originating from the magnetic layer 304. The magnetic particles may take a variety of configurations and structures. In one embodiment, the magnetic particles have a core of the magnetic component, and optionally a polymeric shell whose surface comprises functional groups for linking to a biomolecule. In this embodiment, each of the magnetic particles 306 may have a magnetic core surrounded by an organic or polymeric coating to facilitate the immobilization of biomolecules onto the surface of the particles. In another embodiment, the magnetic particles may comprise a substantially polymeric material with a magnetic material evenly dispersed through the bulk of the particle. In alternate embodiments, the coatings and/or particles may be a biodegradable or non-colloidal. However, each of these embodiments may have in common the fact that the biological molecules may be bound to a detectable label such as a fluorescent marker.

Exemplary of the magnetic component of the magnetic particle that renders the particle magnetic but not able to magnetize other materials are intrinsically magnetic materials such as iron, cobalt, nickel, lanthanides, and the like, either in the free metal form or in the form of a complex, salt, oxide or the like. When particles having magnetic cores and organic or polymeric coatings are used, the cores of the magnetic particles 306 are generally inorganic, and may comprise one or more metals, metal oxides, metal salts, metal hydroxides, alloys of metals, organometallic compounds, and mixtures thereof. The cores may be paramagnetic, ferromagnetic, antiferromagnetic, or ferrimagnetic. Exemplary elemental metals include iron, cobalt, and nickel, but the magnetic cores may also comprise oxides of metals such as ferric oxide, nickel oxide, cobalt oxides, as well as any of the ferrites.

In contrast to a magnetic core with a polymeric coating, each of the magnetic particles 306 may comprise a polymeric matrix into which is impregnated or dispersed a small amount of a paramagnetic or ferromagnetic substance. See, for example, Whitesides, et al., Advances in Biotechnology (1983) 1(5):144-148. Exemplary ferromagnetic substances include iron-based oxides (e.g., magnetite), transition metals, and rare earth elements. The dispersed ferromagnetic substance gives the particle its magnetic properties, allows the particle to be attracted by a magnetic field, and to be captured onto the surface of an array support layer. Similar to the particles described above having a core and coating, however, this type of magnetic particle should provide for an adequate binding surface capacity for the adsorption or covalent coupling of a member of a specific affinity binding pair, i.e., a ligand or a receptor.

As stated above, the nature of the magnetism of the particle, whether it is configured as a core and coating, or dispersed magnetic material within a non-magnetic material, includes paramagnetic, ferromagnetic, antiferromagnetic, and ferrimagnetic properties. In some embodiments, however, the particles are preferably “superparamagnetic”, a characteristic defined herein as a responsiveness to a magnetic field without a permanent magnetization of the particle.

Generally the magnetic particles have an overall density of from about 1.0 to 10.0 g/mL, and preferably a density of from about 1.0 to 5.0 g/mL. Since larger particles are more easily immobilized than smaller particles, larger particles often times do not require as large a magnetic field strength to immobilize the particle as a smaller particle would require. In other words, smaller particles need a stronger magnetic field strength than larger particles for fast and efficient immobilization, and for retaining the particles in their designated positions on the magnetic array substrate 300 during subsequent assay processing.

The magnetic particles 306 may have a wide range of mean diameters. Particles having a mean diameter of from about 0.05 to 1,000 μm can be used, and preferably the particles have a mean diameter of from 2 to 500 μm. The diameters of the magnetic particles will of course have an effect on the surface concentration (or areal density), and thus a wide range of concentrations of magnetic particles on the surface of the array support layer 302 are possible as well. The density size, and surface concentration of the magnetic particles 306 is selected such that the particles are immobilized rapidly and strongly onto the surface of the magnetic array substrate 300 in a desired pattern, and such that their positions remain stable during the assay. For example, particles ranging from about 0.5 to 10 μm are commercially available from Dynal Corporation, Lake Success, N.Y. These particles are composed of spherical polymeric materials into which magnetic crystallites have been deposited. Because of their magnetite content and size, these particles are readily separated in relatively low external magnetic field gradients (0.5 to 2 KGauss/cm).

The magnetic particles 306 may be coated with a variety of materials to which biomolecules are coupled so that the magnetic particles can be used in specific binding assays. Binding of biomolecules to the magnetic particles may be accomplished by any of a number of well-known techniques, widely discussed in the literature. See for example, “Immobilized Enzymes”, Ichiro Chibata, Halsted Press, New York (1978) and “Bioconjugate Techniques”, Greg Hermanson, Academic Press, New York (1996); also Microparticle Reagent Optimization”, Caryl Griffin et al., Seradyn Inc., Indiana (1994); and “Chemistry of Protein Conjugation and Cross-Linking”, Shan Wong, CRC Press, Boca Raton (1991).

The attachment of biomolecules to the magnetic particles 306 may be accomplished chemically by covalent and/or ionic bonding, by physical adsorption, as affinity binding. A wide variety of functional groups are available that may be introduced to the surface of the magnetic particles prior to attachment of the biomolecule. Exemplary functional groups include hydroxyl, carboxyl, cyano, mercapto, ethylene, thiol, amino, aldehyde groups, and the like. In some embodiments, combinations of surface groups are available for binding biomolecules, such as carboxyl (—COOH) or amine (—NH₂). Other surface groups include amides, aliphatic amines, aromatic amines, as well as haloalkyl and hydrazide groups.

In an exemplary embodiment, magnetic particles 306 suitable for use in a magnetic particle array 308 of the present invention preferably have an iron oxide content of approximately 10% to 60% by weight, and a surface —COOH content of between about 20 to 200 microequivalents per gram of magnetic particles 306.

The surface of the magnetic particle may be coated with proteins such as albumin, non-specific immunoglobulin, avidin, fetuin, and so forth, or a carbohydrate such as chitosan, dextran and the like, and of course combinations thereof. Polymeric coatings for magnetic particles include divinylbenzene and polystyrene, or other polymers, copolymers, and terpolymers.

Coating the paramagnetic particles with macromolecules can increase colloidal stability. This can be done by direct adsorption of high molecular weight polymers, or by functionalizing the surface of the particles and then binding macromolecules to the functional groups. Emulsion polymerization and grafting techniques provide a means for coating magnetic particles with polymers.

Functionalized magnetic particles suitable for conjugation to biomolecules are commercially available. Conjugation of biomolecules to magnetic particles has been described in commercial literature, for example, for the conjugation of proteins to amino, carboxyl and epoxy functionalized magnetic particles, the conjugation of carboxyl magnetic particles with biomolecules, and the conjugating of biomolecules with amine and carboxyl functionalized magnetic particles.

Exemplary magnetic particles suitable for use in the various embodiments of the present invention include, but are not limited to, iron oxide particles described in U.S. Pat. Nos. 4,554,088 and 3,917,538; nickel oxide particles described in Biotec. and Bioengr. XIX: 101-124 (1977); Agarose-polyaldehyde beads containing magnetic particles described in U.S. Pat. No. 4,732,811; Dynal beads (commercially available magnetic polystyrene coated beads); Magnogel 44 (magnetic polyacrylamide-agarose beads); and Enzacry (poly-M-diaminobenzene/iron oxide) as described in Clin. Chim. Acta. 69:387-396 (1976). Cellulose containing ferric oxide particles are described in Clin. Chem. 26:1281-1284 (1980) and albumin magnetic microspheres are described in J. Immunol. Methods 53:109-122 (1982). Magnetic porous glass particles are described in WO-A-93/10162. Additional useful magnetic particles and supports for biopolymeric reagents have been described in Robinson et al, Biotechnol Bioeng 15:603 (1973); Pourfarzaneh et al, Methods of Biochemical Analysis, 28:281-3 (1982); and Griffin & Mosbach, App. Biochem and Biotech., 6:283-292 (1981).

Prior to using a magnetic array biochip in an actual assay, the biochip is first prepared by dispensing biomolecular coated magnetic particles 306 onto the surface of the magnetic array substrate 300 in the spatially defined and physically addressable pattern of magnetic particle array 308. Dispensing the particles onto an array may be accomplished by convenient methods known in the art, for example, by inkjet printing, by other types of non-contact mechanical deposition procedures, by contact printing methods such as micro-spotting or micropipetting techniques, and by electromagnetic means. Micropipetting techniques will be discussed briefly since they are simple to implement for almost any type of array, and electromagnetic spotting techniques will be discussed in greater detail since they are so appropriate for spotting magnetic particles.

Conventional micropipetting techniques may be used to spot the magnetic particles 306 into an array 308. A small volume of fluid (less than or equal to about 10 μl) containing the magnetic particles is loaded into the tip of a pipette, and the fluid containing the magnetic particles 306 is then dispensed onto the surface of a magnetic array substrate 300 at a predetermined location (in other words, a spot of the array). It is usually desirable to wash the tip of the pipette prior to the loading of the subsequent sample, which will be spotted onto the array substrate at a different location from the first sample. The process is repeated for each of the samples until eventually the desired magnetic particle array 308 is formed. Of course, the transfer of fluid and magnetic particles may be automated using robotic techniques.

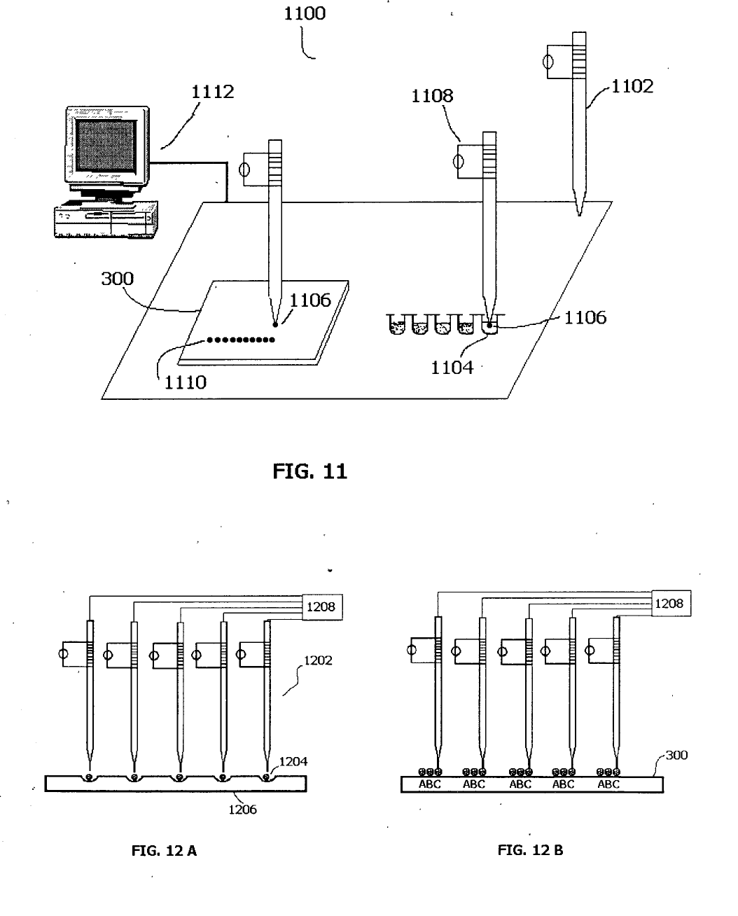

In contrast to conventional micropipette techniques, use of an electromagnetic pin is extraordinarily well suited for spotting magnetic particle arrays. An exemplary system for spotting magnetic particle arrays using an electromagnetic dispenser is illustrated in FIG. 11. Referring to FIG. 11, the system shown generally at 1100 comprising an electromagnetic pin 1102 is immersed in a well 1104 that contains a magnetic particle 1106. The magnetic particle 1106 has a biomolecule (not shown) chemically conjugated to the surface of the magnetic particle 1106. Electric current from a supply and coil 1108 is supplied to the tip of the pin 1102 to magnetize the tip, thus attracting the particle 1106 to the tip of the electromagnetic pin 1102. The magnetic particle 1106 is then transferred to the position on the magnetic array substrate 300 where the particle is to be spotted. The magnetic particle 1106 is released from the tip of the electromagnetic pin 1102 by turning off the current to the pin, and the particle is captured by the magnetic array substrate 300 because the magnetic field from the magnetic array substrate 300 is now stronger than the magnetic field that had been holding the particle to the tip of the pin. Thus the magnetic particle 1106 is released onto the surface of the magnetic array substrate 300 forming a spot on the future array 1110. An advantage of this method is the greater degree of control over the printing of an array because magnetic particles are released and immobilized by regulating the electric current to the tip of the electromagnetic dispenser. A control system 1112 may be used to automate the process.

Cross-contamination between biomolecules is potentially reduced by the use of an electromagnetic dispenser relative to conventional micropipetting techniques (especially the contact type), and no substantial cleaning steps are required after each stage of delivering biomolecule coated magnetic particles to the magnetic array substrate. Also, there is little potential for clogging of an electromagnetic dispensing pin system as seen in some fluid dispensing (e.g., pipetting) systems.

As in micropipette in techniques, multiple samples may be transferred and spotted in a single step by using a set of plurality electromagnetic dispensing pins (as opposed to the single pin shown in FIG. 11). A portion of an electromagnetic system capable of printing multiple spots in a single step is illustrated in FIGS. 12A and 12B. Referring to FIG. 12A, a set of 5 electromagnetic pins shown generally at 1202 is used to withdraw biomolecule coated magnetic particles 1204 from a well plate 1206. Each of the electromagnetic pins of the set 1202 are connected to a controller 1208, and function in a similar manner to the electromagnetic pin of FIG. 11. The controller 1208 causes an electric current to be delivered to the tip of each pin, creating a magnetic field at the tip of the pin which attracts the magnetic particles 1204. In the embodiment illustrated in FIG. 12A there is only one magnetic particle per well of the well plate 1206, but of course there may be more than one magnetic particle per well and this will be discussed shortly. Each of the electromagnetic pins of the set 1202 are spaced apart at a distance which is the same as the spacing (or a multiple of the spacing distance) of the wells in well plate 1206 from which the magnetic particles 1204 are being taken.

The magnetic particles 1204 are transferred to the magnetic array substrate 300 by positioning the set of electromagnetic pins 1202 at the appropriate positions over the magnetic array substrate 300. the controller 1208 turns off the current to the tip of each pin, thus allowing the magnetic field from the magnetic array substrate 300 to attract the particles and immobilize them at their respective positions in the array. Of course, since the spacing between the wells of well plate 1206 is greater than the eventual spacing of the spots in the array, it is necessary to first transfer a set of five magnetic particles (5 in the case being illustrated but there could be more or less than 5 pins in a set) to the positions labeled “A” in the array in FIG. 12B. The electromagnetic pin set 1202 returns to the well plate for a different set of magnetic particles and prints these at “B” on magnetic array substrate 300, where the spacing between printed sets “A” and “B” are much closer that the well-to-well spacing of the well plate 1206. In this manner, an array may be printed with a very high surface density of spots.

Alternatively, a large number of electromagnetic dispensing pins or magnetic transfer probes (5 to 1,000 or more) may be assembled; one for each well of the multi-well plate, and magnetic particles from an entire well plate may be printed onto the magnetic array substrate 300 simultaneously. Printing the array in a single step significantly reduces the time required to build the magnetic array biochip 310. System utilizing magnetic particle transfer probe is illustrated in FIG. 12C.

The electromagnetic dispensing pins are typically made of superparamagnetic materials, such as stainless steel, chromium or platinum, which may be magnetized to attract the magnetic. The pins may also be magnetized with an external magnetic field from a permanent magnet. Simply removing the permanent magnet that had been used to induce the magnetic field will then demagnetize the pin.

The areal density of spots on the surface of the magnetic array substrate 300 is selected to provide adequate resolution of binding events with a probe, particularly in cases where the probe is carrying a variety of different labels. The areal density of spots per array may range in general from 1 to 100,000 or more, including ranges subsumed therein, such as from about 10 to 20,000 and about 100 to 10,000. The density of spots on the surface of the magnetic array substrate may range in general from about 1 to 100 spots per mm² of substrate surface, an in some embodiments will be from about 1 to 20 spots per mm².

The number of magnetic particles per spot in the magnetic particle array 308 is also selected to provide sufficient detection sensitivity of binding events between targets and probes. Depending on the label, the size of the magnetic particle, the assay format selected, and other factors, the number of magnetic particles per spot will generally range from about 1 to 10⁸, and ranges subsumed therein, such as from about 1 to 10³ particles per array spot, and from about 1 to 50 particles per array spot. A single magnetic particle 306 may have one or more biomolecular moieties attached to its surface. Similarly, each spot may contain same biomolecular moiety attached magnetic particles or may contain mixtures of magnetic particles each with at least one unique biomolecular moiety attached to its surface.

By varying the operating parameters of the dispensing system, i.e., the size of the magnetic particles, the number of magnetic particles per spot, the pattern of magnetic poles in the magnetic array substrate 300, the array spot size can be controlled such that spots of various dimensions may be produced. The sizes of the spots can have widths (which for a round spot would be its diameter) that are in the range of from about 5 μm to 5 mm. In embodiments where very small spot sizes are required, materials may be selected accordingly to provide small spots whose width is in the range of about 1 μm to 1 mm, including ranges subsumed therein, such as 25 to 5,000 μm.

The pattern of the array may conform to a variety of different geometries, ranging from orthogonally organized rows and columns, grids, curvilinear rows across the substrate surface, concentric circles or semi-circles, or simply rows of lines and the like. According to certain embodiments, there may be a plurality of identical arrays across the surface of the substrate. Each array may contain multiple regions having the same type of array spot, or different types of array spots. The number of discrete regions on a single array may range from 10 to 5,000, although more or less are possible.

Analytical devices based on magnetic array biochips may be mass produced by techniques that include lasering, embossing, injection molding, reaction injection molding, casting, compression molding, Lithographie Galvanoformung Abformung (LIGA), electroplating, and electroforming. The devices may also be manufactured by methods used by the semiconductor industry, including photolithography, reactive ion etching, ion beam milling, casting, and micromachining. Alternatively, magnetic array devices may be fabricated by printing techniques such as serigraph printing, lamination, ink jet printing, offset press printing, thermal laser printing, silk screening, intaligo printing, flexography, gravure printing, and lamination. It will be understood that the methods utilized to manufacture magnetic array biochips and devices containing magnetic array substrates according to aspects of the present invention are not critical.

Materials within the magnetic array biochip that comprise polymeric materials include, but are not limited to polyolefins such as polypropylene and polyethylene, polyesters such as polyethylene terephthalate, styrene containing polymers such as polystyrene, styreneacrylonitrile, and acrylonitrilebutadienestyrene, polycarbonate, acrylic polymers such as polymethylmethacrylate and poly acrylonitrile, chlorine containing polymers such as polyvinylchloride and polyvinylidenechloride, acetal homopolymers and copolymers, cellulosics and their esters, cellulose nitrate, fluorine containing polymers such as polyvinylidenefluoride, polytetrafluoroethylene, polyamides, polyimides, polyetheretherketone, sulfur containing polymers such as polyphenylenesulfide and polyethersulfone, polyurethanes, silicon containing polymers such as polydimethylsiloxane. In addition, the structures can be made from copolymers, blends and/or laminates of the above materials, metal foils such as aluminum foil, metallized films and metals deposited on the above materials, as well as glass and ceramic materials.

Suitable methods for constructing/fabricating the magnetic substrate and capillary related devices of the present invention are described in U.S. Pat. Nos. 6,074,725, 6,167,910, 6,182,733, 6,176,962, and 6,129,854.

The magnetic array biochips of the present invention find use in a variety of applications, where such applications generally involve the detection of analytes. The term “analyte” refers to the compound or composition to be detected or measured, and which has at least one epitope or binding side. The analyte can be any substance for which there exists a naturally occurring binding member or for which a binding member may be prepared. Analytes include, but are not limited to, toxins, organic compounds, proteins, peptides, microorganisms, amino acids, carbohydrates, nucleic acids, hormones, steroids, vitamins, drugs (including those administered for therapeutic purposes as well as those taken for other purposes), virus particles and metabolites of virus particles, or antibodies to any of the above substances. Detection of an analyte in a given sample may be detected at least qualitatively, if not quantitatively.

Generally, the sample suspected of comprising the analyte of interest is contacted with the magnetic particle array of a magnetic array biochip produced according to the methods discussed herein. The sample is flowed through the magnetic array biochip under conditions sufficient for the analyte to bind to its respective binding pair member that is present on the biomolecules associated with the magnetic particle array. Thus, if the analyte of interest is present in the sample, it binds to the array at the site of its complementary binding member and a complex is formed on the array surface. The presence of this binding complex at specific sites of the array surface is then detected through the use of a signal production system, and thus the presence of analyte in the sample is deduced.

In general, the steps of a typical assay using a magnetic array substrate include:

a) providing an magnetic array substrate 300;

b) conjugating a target molecule(s), e.g., biomolecule(s) to a magnetic particle(s) 306, each target molecule capable of binding with a specific complementary member e.g., analyte (also called a probe), and/or components of a specific binding member;

c) forming an array by micro-spotting, printing, or otherwise transferring the magnetic particles 306 onto the surface of the magnetic array substrate 300 to immobilize the conjugated molecules into a spatially defined magnetic particle array 308;

d) forming a composition containing a sample with a labeled analyte, the sample being capable of binding with a biomolecule immobilized in the magnetic particle array 308;

e) treating the magnetic particle array 308 with the labeled analyte;

f) incubating the magnetic particle array 308 with the composition containing the sample to form a complex which includes the labeled analyte;

g) washing the magnetic particle array 308, if required, to remove unbound analyte (and label) that did not bind to target biomolecule;

h) inducing the labeled analyte and immobilized biomolecule complex to produce signal; and

i) measuring the signal to indicate the presence of the analyte of interest in the sample as a function of position in the array.

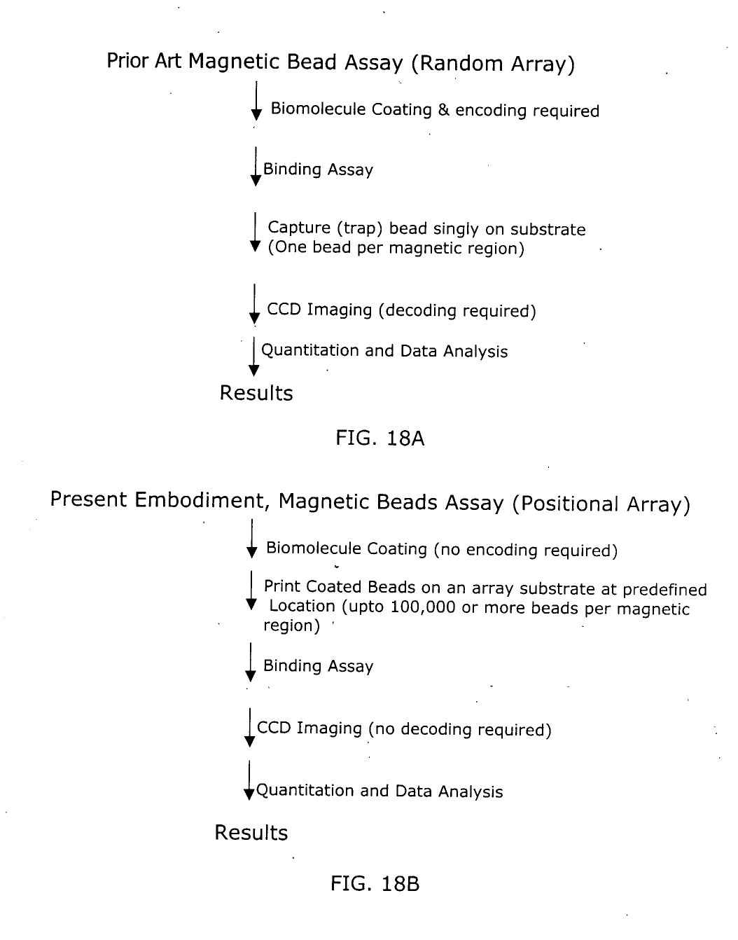

FIG. 18B is a representative flowchart showing embodiment of a method of the present invention.

Following the printing of the magnetic particles 306 onto the surface of the magnetic array substrate 300 to form the magnetic particle array 308, the resultant array may be used in the “as is” configuration at this point, or it can be incorporated into a biochip, multi-well or other device and conveniently stored for use at a later time. Under appropriate conditions, the biomolecular arrays may be stored for 6 months to 1 year or longer. The arrays may be stored at a temperature within the range of about −20 degrees Celsius to room temperature. Arrays that have been prepared but not yet assayed may be sealed in rigid plastic (or some other type of) container, and preferably shielded from heat, light, humidity and external magnetic field sources, etc.

The same types of assays that may be conducted using conventional techniques (where the biomolecule is chemically conjugated to the surface of a substrate to hold the biomolecules positionally in an array format) may be done using a magnetically immobilized array. Representative assays for which magnetic immobilization are appropriate have been described in Diagnostics in the Year 2000, edited by P. Singh, B. Sharma and P. Tyle, Van Nostrand Reinhold, New York, 1993; Immunochemical Assays and Biosensor Technology, edited by R. Nakamura, Y. Kasahara and G. Rechnitz, μmerican Society for Microbiology, Washington, 1992; Microarray Biochip Technology, edited by Mark Schena, Eaton publishing, 1998; and DNA Microarrays: A Practical Approach, edited by Mark Schena. Oxford University Press, 2000.

Some of the binding reactions and assay formats that can be utilized by the present invention include antigen-antibody reactions, nucleic acid hybridizations, enzyme-substrate binding, ligand-receptor binding, and binding reactions between biotin-streptavidin, carbohydrate-lectins, DNA-antibody, metal-chelate, and the like. It is understood that any person having skill in the art may formulate a desired binding protocol and assay format using the magnetic particle arrays 308 of the present invention.

Although the use of a magnetic particle array 308 is applicable to virtually any type of assay, a brief overview of a DNA assay and an exemplary antibody binding assay will given, and since these are common types of assays, and because they are particularly suitable to magnetic particle arrays. Typically, in a DNA assay, once the immobilized DNA array is fabricated, it is exposed to a hybridization reaction. The nomenclature to be used here will refer to the DNA sequence immobilized on the magnetic array substrate 300 as “the target,” and the DNA sequence transferred through the sample inlet port 312 as “the probe.” The probe is isolated from sample biological material, amplified, and labeled with a suitable marker group such as a fluorescent or luminescent label. The labeled probe is then incubated with the magnetic particle array 308 under hybridization conditions using appropriate fluidics and hybridization ovens. During hybridization the labeled probe binds to target. After the hybridization reaction is complete, the magnetic particle array 308 is inserted into and imaging scanner, where the array spots were hybridization was successful are detected. Probes that most clearly match the target produces stronger signal than those that have mismatches. Since the sequence and the position of each target on the array is known, by complementarity, the identity of the sample nucleic acid may be determined.

Similarly, in the case of antibody binding assays, the magnetic particle array 308 is incubated with sample containing the analyte, and the labeled substance capable of forming the complex. After the incubation is complete, the array may be washed to remove unbound components. The array may then be read by and imaging device such as a fluorescent scanner or other means, and at each location of the array the presence and quantity of the analyte may be determined.

The reaction between a target and a probe (for example, a hybridization reaction in the case of a DNA assay) usually involves contacting the array with the labeled probe with an aqueous medium. Contact may be achieved in a variety of different ways depending on the specific configuration of the magnetic array biochip 310 and the type of assay for which the magnetic particle array 308 is being applied. For example, where the magnetic particle arrays 308 comprises a pattern of magnetic particles immobilized on the surface of a plate-like or microscopic slide substrate, which may include rigid substrates, contact may be accomplished by simply placing the magnetic array substrate 300 in a container comprising the labeled probe solution. The container may be a tray, dish, and the like. In other embodiments, the magnetic particle array may be incorporated into a biochip device having fluid entry and exit ports. In this case, the probe solution may be introduced into the chamber through the sample entry port 312, and the fluid may be transported either manually, or with an automated device. In multi-well embodiments, the labeled probe solution will be introduced in the reaction wells holding the array, again either manually, such as with a pipette, or with an automated fluid handling device.

The time of contact between the immobilized target and labeled probe varies depending on the type of assay, but will of course be maintained for a sufficient period of time to allow the binding to occur. Contact will generally be maintained for a period of time ranging from about 1 minute to 24 hours or more, often from about 2 minutes to 12 hours, and usually from about 10 minutes to 6 hours.

The uses of the magnetic particle arrays of the present invention in biological and medical assays are not limited by the type of label chosen for the assay. A variety of labels may be employed in association with the probes of the present invention including fluorescent marker, luminescent, enzymatic, cofactor, dye, particle, phosphorescent, metal-chelate, spin, metal sol, radioactive, heavy metal, electroactive, quantum dot particles. Following binding of the probe to the target, the resultant patterns of labeled probe-target complexes may be visualized, imaged, or detected in a variety of ways. Representative detection means include scintillation counting, autoradiography, fluorescence measurements, luminescence measurements, and the like.

Since it is possible that not all the label that is exposed to the magnetic particle array 308 will be bound to the target biomolecules immobilized on the magnetic particles 306, it may be necessary, in some embodiments, to wash the excess label off prior to the detection step. The need for a non-bound label removal step prior to the detection step may in some embodiments depend on the particular label employed by the assay. For example, in homogenous assay formats a detectable signal is only generated upon specific binding between probe and target. As such, in homogenous assay formats, the binding pattern may be detected without a non-bound label removal step. In other embodiments, the label will generate a signal whether or not the label is bound, and in these cases, it is advantageous to remove the non-bound labeled probe prior to the detection step. One way of removing the non-bound labeled probe is by means of a common washing step, where a variety of solutions and protocols for their use are known to those of skill in the art.

The assay methods described above may be modified for multiplex analysis. For example, one may employ a plurality of different probes that are each distinguishably labeled. It is also conceivable that each magnetic particle and /or array spot has its own uniquely distinguishable and detectable marker (label) that allows identification of the spot position and/or the immobilized biomolecule it carries. This has the advantage that the precise location of each array spot or address location is identifiable by the label, rather than by having recorded its position. In embodiments of the present invention, the magnetic particle array 308 (and associated magnetic array biochip 310, if the array has been so packaged) may be provided in kit form for performing the binding assays described above in field conditions. The kits may have self-contained fluid sources filled with reagents for use in the assay, where the reagents include all manner of fluids, such as amplification reagents, biomolecule conjugation reagents, sample treatment reagents, hybridization buffers, signal producing labels, etc.

Finally, a complete magnetic array biochip system includes such components as a controller, to imaging means, pumps and reservoirs for the microfluidic hardware, and information processing means. By the term “system” is meant the working combination of the enumerated components. Systems of the subject invention will generally comprise the array, a fluid handling device capable of contacting the probe fluid and reagents with the target molecules on the array, and means for delivery and removal of wash fluid from the array surface; a reader which is capable of providing identification of the location of positive probe target binding events and the intensity of the signal generated by such binding events. The controller may be a computer which is capable of controlling the actions of the various elements of the system, including the time at which the reader is activated, the time at which the sample fluid is introduced, etc.

In general, bioassays using magnetic particle arrays offer the advantages of being less variable and more flexible than conventional bioassays. Furthermore, assays using magnetic particle arrays may be capable of higher throughputs than conventional assays.

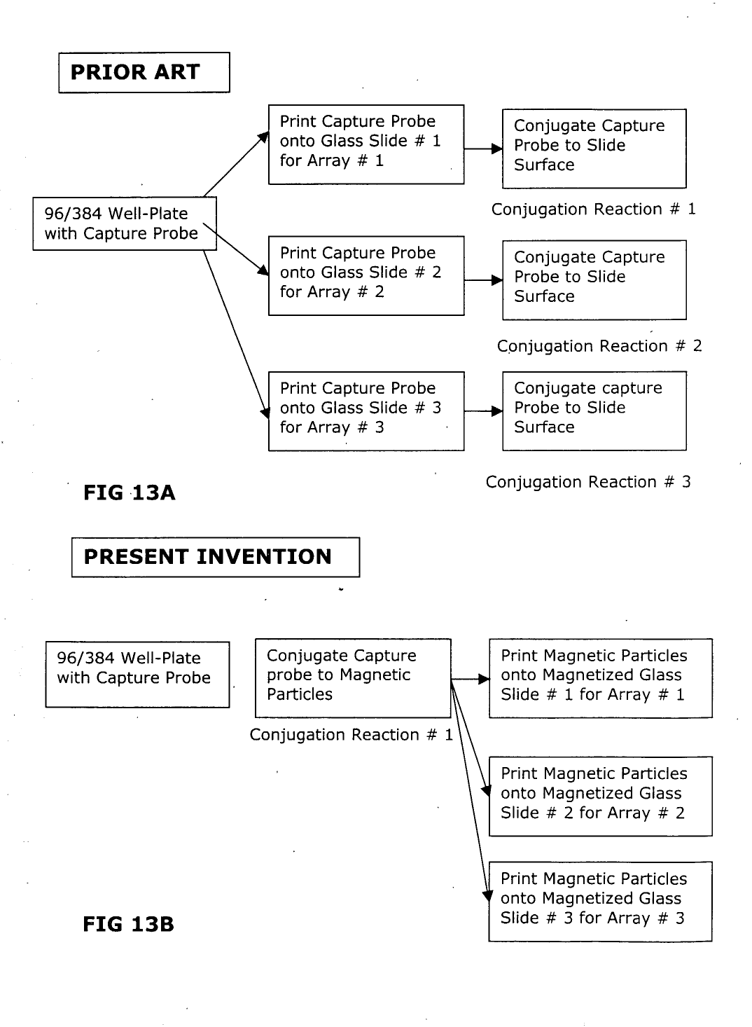

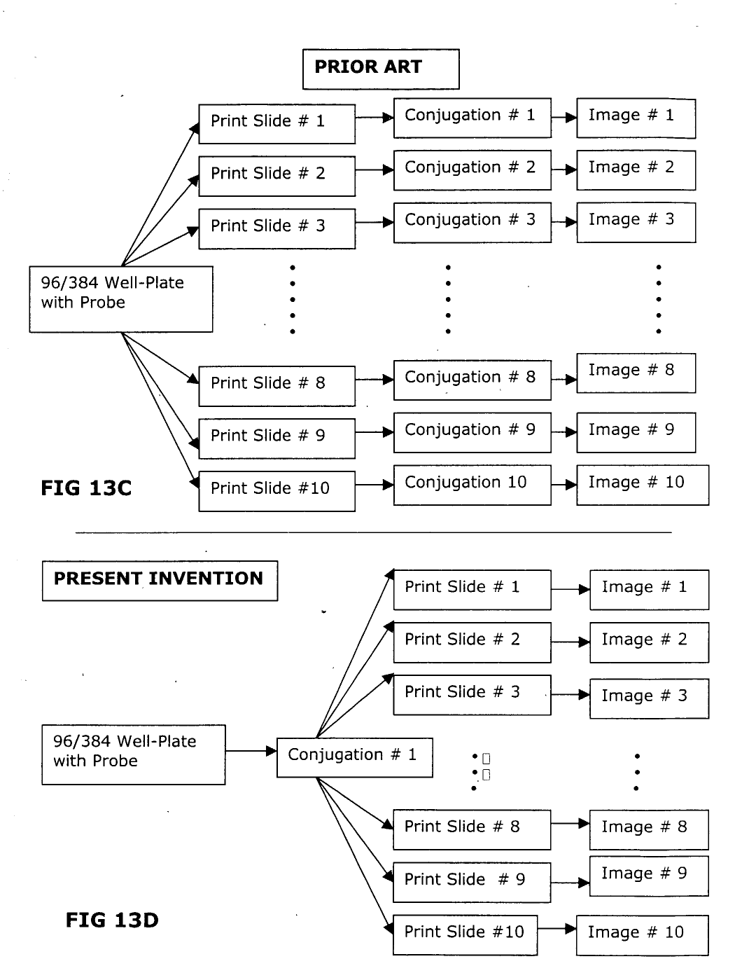

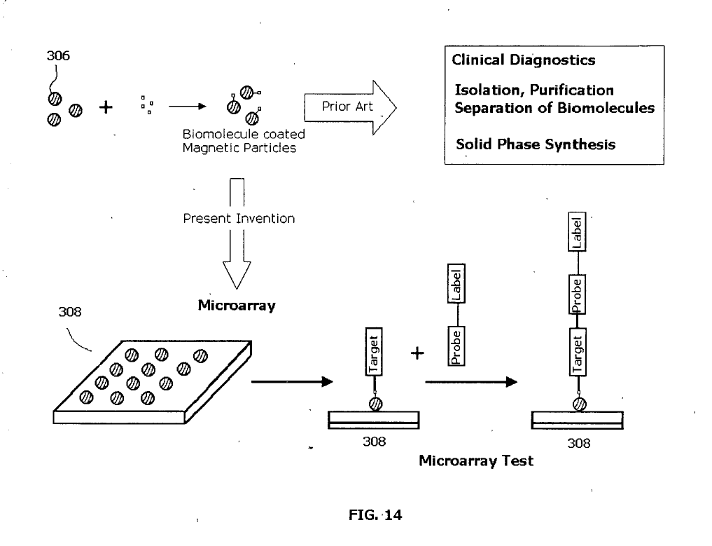

An example of how the magnetic particle array 308 is able to produce less variable results in an assay (in other words, provide more repeatable and reliable results) is illustrated in FIGS. 13A-D. These figures show conventionally how a separate conjugation reaction is required for each assay, whereas in embodiments of the present invention only one conjugation reaction is needed for each of the assays to be performed. Conventionally, as shown in FIG. 13A, a well plate provides a patient's target sample for two different tests labeled #1 and #2. For test #1, a target sample is removed from the well plate and printed onto a substrate (which may be a glass microscope slide). The target is conjugated to the slide surface in conjugation reaction #1. The probe and tag is then introduced to the array spot and imaged for test #1. A similar sequence of events is performed to print and conjugate the patient's sample onto a second substrate for test #2 and third substrate for test #3.

FIG. 13A shows that a separate conjugation reaction is required for each test. A variability may be introduced into the assay with this method because even though the conditions of the different conjugation reactions may be substantially the same, variations may nonetheless exist.

In contrast, embodiments of the present invention may provide less variability because conjugated biomolecules from the same conjugation reaction may be used in multiple tests. Referring to FIG. 13B, a patient's target sample is conjugated onto the surface of the magnetic particles of the present invention. This represents the first and only conjugation reaction that may be necessary. For example, some of these magnetic particles may then be printed onto a first substrate for test #1. If it is desired to conduct a second test, some of the remaining conjugated magnetic particles from the same lot (denoted conjugation reaction #1) may be printed onto a second substrate, perhaps at a later time, for test #2. In other words, the same collection of conjugated biomolecules may be used in both tests, thus reducing the variability of the assay, which may otherwise have arisen from separate, individual conjugation reactions. By limiting the assay protocol to a single conjugation reaction, and then using conjugated biomolecules from that lot as needed, some of the variability of the assay will have been eliminated.

This concept may be illustrated more dramatically in FIGS. 13C-D, which emphasizes the potentials advantages for a 10-test example. In the conventional assay illustrated in FIG. 13C, 10 different conjugation reactions are necessary, one for each of the 10 tests to be conducted. Even greater variability may occur in the 10-test example of FIGS. 13C-D (relative to the 2-test example of Figures A-B) since the errors that result from minor differences in the conjugation conditions are now accumulated over 10 reactions. In contrast, FIG. 13D illustrates an embodiment of the present invention where one conjugation reaction takes place, and the printing and imaging steps for each of the 10 tests are conducted using conjugated biomolecules from that single conjugation reaction. The potential variability arising from different conjugation steps has been eliminated.

To summarize: a separate conjugation reaction may be necessary for each of the tests conducted with conventional assaying techniques, whereas according to embodiments of the present invention, a single conjugation reaction may be performed to feed a number of subsequent tests.

In a corollary of the previous example, a single lot of biomolecules conjugated to magnetic particles may be used in both a microarray assay, as well as in tests that are part of a clinical diagnosis. For example, as shown in FIG. 14, a target biomolecule (denoted by the square symbols) may be conjugated to magnetic particles 306 to be used in a clinical diagnostic test. If the test is negative, nothing further may need to be done. On the other hand, should the clinical diagnostic test prove to be positive, conjugated biomolecules from the same lot may be used in a subsequent microarray assay. Since the conjugated biomolecules of the assay came from the same lot as those used in the clinical diagnostic test, a source of variability has been eliminated.

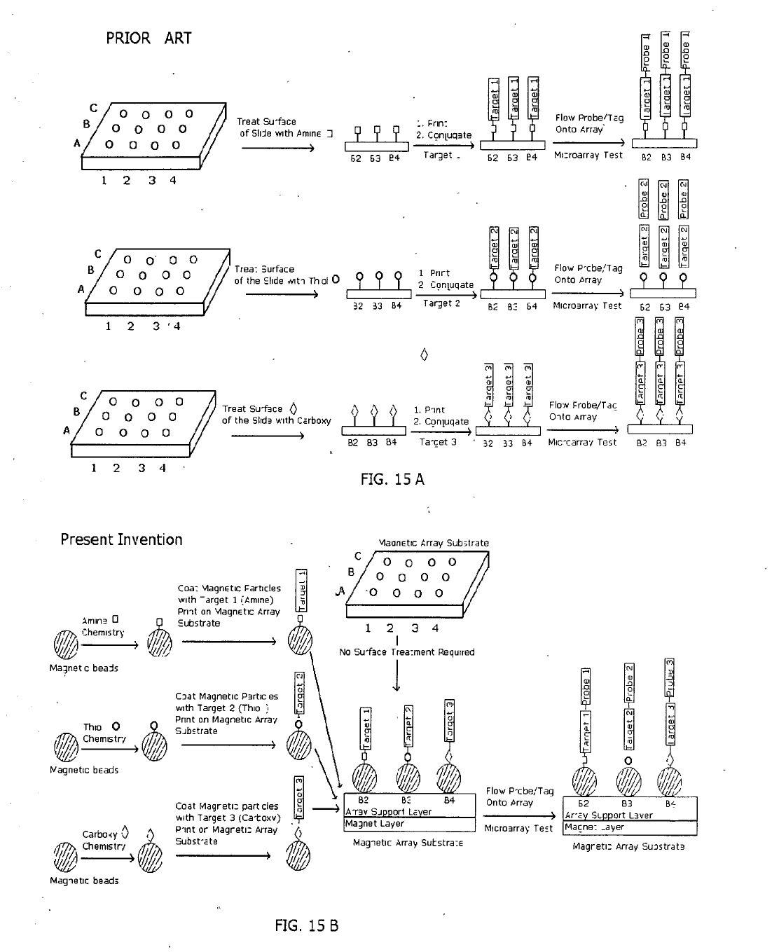

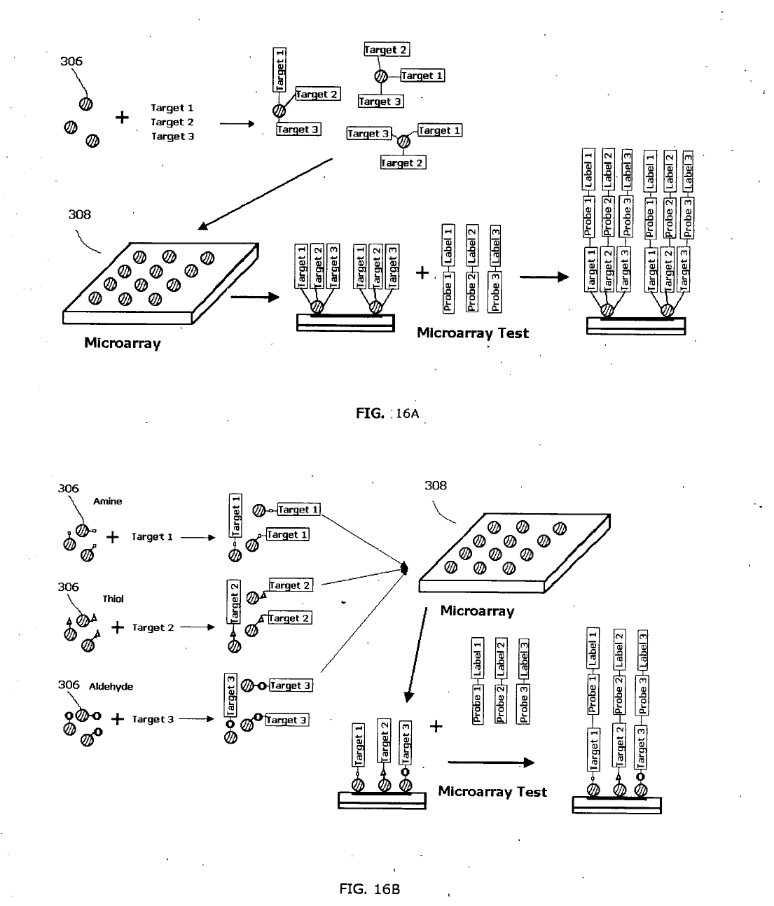

Magnetic particle arrays provide more flexibility than conventional, non-magnetic arrays. This is due in part to the fact that magnetic particles provide a several-fold larger surface area for immobilization of the biomolecules of interest compared to the area which each magnetic particle projects onto the surface, and the increased number of biomolecules may be immobilized per unit surface area of array substrate surface, increasing the sensitivity of the assay, and the target capture efficiency. Moreover, since the magnetic particles 306 provide a three-dimensional structure for biomolecule binding, the reduced stearic hindrance effect may result in increased binding kinetics. An example of how the magnetic particle array 308 is able to provide greater flexibility than conventional arrays is illustrated in FIGS. 15A-B.

Referring to FIG. 15A, a conventional assay may involve treating the entire surface of an array substrate (which may be a glass microscope slide) with an amine chemistry (shown in FIG. 15A as a square symbol). An amine group is attached to each of the array positions of the substrate, shown in cross-section at just three of the positions of the array that have been identified as positions B2, B3, and B4. A biomolecule labeled target 1 is then printed onto each of the array positions such that it is conjugated to the substrate through the amine groups. A sample probe 1 is then flowed over the array such that probe(s) 1 may react chemically with complementary biomolecules it finds, which in this case will be at those positions having a target 1 conjugated to the array surface. Since probe 1 has a preattached tag 1, the presence of a complementary binding at the array spots B2, B3, and B4 may be detected.

In a similar manner, an assay that requires a thiol group for conjugation (shown in FIG. 15A as a circle symbol) may be attached to the surface of a second array substrate, and an assay requiring a carboxy group for conjugation (shown in FIG. 15A as a diamond symbol) may be attached to the surface of a third array substrate. Probe 2 binds to complementary biomolecule target 2 on the second array substrate, and probe 3 binds to complementary biomolecule target 3 on the third array substrate.

In the exemplary conventional assays of FIG. 15A, the entire surface of an array substrate is treated with only one type of conjugation group. This may be accomplished by dipping the substrate into a chemical solution of the conjugating group. Although it may be possible to attach different conjugating groups to different positions on the same substrate (such as an amine group at position B2 and a carboxy group at position B3 on the first substrate), this requires very precise and complicated printing technologies. In general, those skilled in the art investigate only one type of target-probe reaction on each substrate array.

In contrast, the enhanced flexibility of the present invention may be shown schematically in FIG. 15B, where magnetic particles 306 are treated with the chemical groups (that will eventually provide conjugation to the array substrate), in an “off-line” manner, meaning that the treatment is not done in the presence of the array substrate. As shown in FIG. 15B, no surface treatment of the array substrate is necessary. The magnetic particles coated with the conjugating groups may be mixed with the solution of the target sample to form a suspension such that target 1 is conjugated to any magnetic particle having an amine group (square symbol), target 2 is conjugated to magnetic particles through thiol groups (circles), and target 3 and is conjugated to magnetic particles through carbohydrate groups (diamonds). The magnetic particles may then be printed onto a magnetic array substrate 300 without regard to chemistry, because conjugation specific chemistry has already been done off-line. In the exemplary assay of FIG. 15B, target 1 is attached to position B2, target 2 is attached to position B3, and target 3 is attached to position B4. In this manner, it is possible to assay for the binding of probe 1 to target 1, probe 2 to target 2, and probe 3 to target 3 on the same array substrate. The detection of such binding events may be accomplished by separately imaging tag 1, 2, and 3 respectively, which may be differentiated (for example) through different florescent wavelengths. No complicated printing technologies were required in this example because the magnetic particles allowed the conjugation process to be performed off-line.

A further example of the flexibility offered by embodiments of the present invention is illustrated in FIG. 16A, which shows how multiple types of targets may be conjugated to a single magnetic particle, and how multiple magnetic particles (with either the same or different types of targets) may be immobilized at one array spot. In the specific example of FIG. 16B, amine (squares), thiol (circles) and carbohydrates (diamonds) are attached to magnetic particles. Any individual magnetic particle may have one, two, or more different types of conjugation chemistries on its surface. Because target 1 conjugates only to the amine chemistry, target 2 to the thiol chemistry, and target 3 to the carbohydrate chemistry, respectively, each magnetic particle may have more than one type of target conjugated to it. As before, probe 1 binds to target 1, probe 2 to target 2, and probe 3 to target 3, respectively. Thus, a position B3 on the array may display during imaging tags 1, 2, and 3. This type of flexibility is not readily available with conventional techniques.

Embodiments of the present invention may offer advantages of higher throughput over conventional arraying techniques. In some conventional, contact-printing technologies that use microfluidic and pin printing techniques, a pin having a reservoir is used to transfer a small amount of liquid from a microtiter plate to the slide on which the array is printed. The sample is drawn into the tip of the pin where the reservoir is located, and after that small quantity of liquid is printed on the slide, the pin is washed and dried in a vacuum. In some cases, it may take as long as 1.6 hours to process a 384-well microtiter plate.

According to embodiments of the present invention that use electromagnetic pins to print magnetic particle arrays, it is not necessary to carry out as thorough a washing step because the sample contacts the magnetic particles, and not necessarily the magnetic pins. Alternatively, the magnetic particles may be disposed of after an assay, or washed off-line, so that throughput is not affected by the washing step.

Additionally, no pre-printing is necessary with the present invention. In conventional contact printing technologies, the contact printing pins are dipped into the wells of the microtiter plates supplying the sample fluids for the assay. If the volume of the sample fluid exceeds about 6 microliters per well, the pins must be pre-spotted to drain off excess liquid from the exterior of the pin. Only when the excess liquid is removed will consistent spot sizes be printed on the substrate. Since the biomolecule is pre-attached to the magnetic particle before spotting, there is no equivalent microfluidic loading step in embodiments of the present invention, and there is less variability in a “spot size” of the sample, per se, because the spot size may simply be the diameter of the (group of) magnetic particle(s). Since no preprinting is necessary with the present invention, throughputs are improved.

In summary, then, throughputs may be improved because magnetic pins do not necessarily need to be washed and dried; magnetic particles can be washed and dried off-line; magnetic particles may be disposable; conjugation targets to magnetic particles may be done off-line; and pre-printing may not be necessary.

Embodiments of the present invention offer a number of miscellaneous advantages over conventional arraying techniques. Magnetic particles provide a several fold increase of surface area for the immobilization of biomolecules, relative to a comparable flat surface. The increase in the number of biomolecules that may be immobilized on the surface of the magnetic particle usually leads to an increase in the sensitivity of the assay, and an increase in capture efficiency. Moreover, magnetic particles provide a three-dimensional structure for biomolecule immobilization, leading to reduced stearic hindrance effects when probe molecules bind to target biomolecules. In some embodiments, a porous structure of the surface of a magnetic particle may lead to an increased surface area on the surface of the particle, and a potentially enhanced binding capacity per particle. For example, porous MagneSil™ particles, supplied by Promega Corporation have a surface area of 27 m²/g, as compared to 8 m²/g for non-porous particles.

Magnetic particles are compatible with number of organic and aqueous solvents, salts and are stable at elevated temperatures necessary in hybridizations reactions. Since magnetic particle lend themselves for off-line coupling with biomolecules, coated magnetic particles can be tested independently to ascertain the quality of the biomolecule immobilization, and hence the quality of the microarray may be predicted before printing. Alternatively, coated magnetic particles can be tested following the assay. An additional advantage of magnetic particle arrays is that by controlling the number of particles per spot, a user may manipulate and optimize the signal-to-noise ratio, and thus the sensitivity, and specificity of the assay.

Magnetic particles have been used and are compatible with number of different labels and detection chemistries such as enzymes, fluorescent, chemiluminescent, bioluminescent, electrochemiluminescent, electroactive labels and the likes. Further, magnetic particles lend themselves to multi-analyte assay formats. Magnetic particles can be coated with polymer containing various concentrations dye to produce hundreds and potentially several thousands of unique magnetic particles. The specific dye proportions permit each color-coded spots to be readily identified based on its fluorescent signature thus, allowing large number of analysis to be performed in single sample. This would allow microarray experimentation without specific prior knowledge of the precise array site address of specific spot.

In the present invention, magnetic beads are magnetically deposited/immobilized onto the magnetic array substrate in spatially defined and physically addressable manner to form positional magnetic bead array. Since the physical address of the array spot (and of bead) is known, encoding and decoding of individual bead is not required to practice this invention. FIG. 18 B is representative of flowchart showing the assay method of the present invention. Signal from individual magnetic site containing beads is measured simultaneously. Since beads are positionally fixed, commonly available readers can be conveniently used to detect and analyze the results in the present invention. The dimension of the magnetic region in magnet layer is not dependent on the size and shape of the magnetic particle used for the assay. Furthermore size of the magnetic region in the present invention is several time larger than the size of the particle used and magnet layer can be fabricated any number of commonly available methods including injection molding, compression molding, calendering, casting, extrusion, printing techniques, spin coating and vapor deposition. Strength of the magnetic region is significantly higher in the present invention as several beads can be immobilized per magnetic region ( up to 106 beads per magnetic region) unlike single bead per magnetic region. In the prior art. Uniformity in the size and shape of the magnetic particle is not critical in the present invention compared to prior art. The present invention combines the best of bead and positional microarray technologies as illustrated in Table 1.

TABLE 1
Advantages of Positional Magnetic Particle Array

| | | | | Multiple | Manufacturing | Use of | |
|---|---|---|---|---|---|---|---|
| Feature | | Chemical | | Sample | Turnaround of | Existing | |
| Technology | Reproducibility | Flexibility | Scalability | Analysis | Short Runs | Readers | Customizable |
| Positional | | | | | | | |
| Array | | | | | | | |
| 1) Spotted | Moderate | Low / | Low/ | Low | Slow | Yes | Moderate |
| Arrays and | | Moderate | Moderate | | Moderate | | |
| 2) Synthe- | | | | | | | |
| sized Arrays | | | | | | | |
| 3) Present | High | High | High | High | Rapid | High | High |
| Invention- | | | | | | | |
| Magnetic | | | | | | | |
| Bead Arrays | | | | | | | |
| Random | High | High | Moderate | High | Rapid | No | High |
| (virtual) | | | Requires | | | Requires | |
| Arrays | | | Encoding & | | | Special | |
| Bead Arrays | | | Decoding | | | Readers | |

Unless defined otherwise, all technical and scientific terms used herein have the same meaning as commonly understood by one of ordinary skill in the art to which this invention belongs.

Although any methods and materials similar or equivalent to those described can be used in the practice or testing of the present invention, the preferred methods and materials are now described.

All publications and patent documents referenced in this application are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

I claim:

1. A magnetic particle array for performing biological and chemical assays, the magnetic particle array comprising:
at least one magnetic layer comprising a predetermined spatial distribution of spaced-apart magnetized spots;
a plurality of magnetic particles immobilized to said magnetic spots;
wherein at least a plurality of said particles comprise different molecules.

2. The magnetic particle array of claim 1 further comprising:
an array support layer to which said magnetic layer is attached.

3. The magnetic particle array of claim 2, further comprising a biochip holder for supporting
the magnetic particle array, wherein the biochip holder contains at least one fluidic channel for transporting a reagent(s) to the magnetic particle array.

4. The magnetic particle array of claim 3, wherein the biochip holder further comprises an inlet port for introducing a reagent to the magnetic particle array via the fluidic channel.

5. The magnetic particle array of claim 3, wherein the biochip holder further comprises a reaction chamber adjacent to the magnetic particle array.

6. The magnetic particle array of claim 1, wherein said different molecules comprise one or more molecules from group consisting of, but not limited to, chemical molecules, polymers, biopolymers, proteins, peptides, cells, enzymes, substrates, antibody, antigen, bacteria, virus, hapten, drugs, receptors, recombinant molecules, DNA, RNA, PNA, Poly A, oligonucleotides, carbohydrates, hormones, metal sol, metal chelates, dyes, particulates, label moiety such as fluorescent or luminescent molecules, member of specific binding reaction and the like.

7. The magnetic particle array of claim 2, wherein the array support layer is made from a polymer or combination of polymers, plastic,, silicon, resin, polysaccharide, silica, hydrogel, carbon, metal, inorganic glasses, membrane, porous or non-porus material, and paper-based material.

8. The magnetic particle array of claim 2, wherein the surface of the array support layer contains a flexible or rigid structure(s) selected from the group consisting of: planar, irregular, a well, grooves, multi-well plate, dish, screen, mesh, depression, elevation, trench, chamber, nanowells, array of pits, strips, dipstick, fluidic network, capillary, porous film, non-porous film, gel and channels.

9. The magnetic particle array of claim 2, wherein the array support layer has a surface treated or coated, derivatized or modified with chemical or polymer or biopolymer.

10. The magnetic particle array of claim 1, wherein the at least one magnetic layer comprises a material selected from the group consisting of:, but not limited to, a fine magnetic powder loaded into a thermoplastic binder; a bonded material comprising neodymium, iron, and boron;; a sheet of plastic material impregnated with a ferromagnetic material; and a sheet of synthetic resin material having mixed therein magnetic powder particles, neodymium-iron-boron, samarium-cobalt, barium ferrite, and strontium ferrite.

11. The magnetic particle array of claim 1, wherein the at least one magnetic layer has a surface treated or coated, derivatized or modified with chemical or polymer or biopolymer.

12. The magnetic particle array of claim 1, wherein said magnet layer has one dimension in the range of from about 0.0001 to 0.5 inches.

13. The magnetic particle array of claim 1, wherein said magnetic layer has a magnetic field strength in the range of about 100 to 15,000 Gauss.

14. The magnetic particle array of claim 1, wherein said magnetic layer is patterned permanent magnet.

15. The magnetic particle array of claim 1, wherein said magnetic layer is electromagnetic.

16. The magnetic particle array of claim 1, wherein said magnetic layer, is produced by any number of methods including; extrusion, calendering, injection molding, compression molding, printing, spin coating, chemical or vapor deposition, sputtering and combinations thereof.

17. The magnetic particle array of claim 1, wherein said magnetic particles comprise a magnetic core surrounded by an organic or polymer coating.

18. The magnetic particle array of claim 17, wherein the magnetic core of the magnetic particles is selected from the group consisting of one or more metals, metal oxides, metal salts, metal hydroxides, alloys of metals, organometallic compounds, and mixtures thereof.

19. The magnetic particle array of claim 1, wherein magnetic particles are swellable

20. The magnetic particle array of claim 1, wherein magnetic particles are non-swellable

21. The magnetic particle array of claim 1, wherein the mean diameter of the magnetic particles ranges from about 0.05 to 1,000 μm.

22. The magnetic particle array of claim 1, wherein the areal density of magnetic particle array spots on the surface of the array support layer ranges from about 1 to 100 spots per mm².

23. The magnetic particle array of claim 1, wherein the number of magnetic particles per array spot ranges from about 1 to 10⁶.

24. The magnetic particle array of claim 1, wherein the total number of spots in the array ranges up to about 100,000.

25. The magnetic particle array of claim 1, wherein the largest dimension of each of the array spot ranges from about 1 um to 5 mm.

26. The magnetic particle array of claim 1, wherein the pattern of spots in the array is selected from the group consisting of orthogonally organized rows and columns, grids, curvilinear rows across the substrate surface, concentric circles, concentric semi-circles, and simple rows of lines.

27. The magnetic particle array of claim 1, wherein said different molecules are attached to magnetic particles prior to forming magnetic particle array.

28. The magnetic particle array of claim 1, wherein said different molecules are attached to magnetic particles after forming magnetic particle array.

29. The magnetic particle array of claim 1, wherein each magnetic array spot contains magnetic particles coated with identical or mixture of different molecules.

30. The magnetic particle array of claim 1, wherein each array spot contains one or more type of magnetic particles, wherein each particle type is distinguishable

31. A magnetic particle array as set forth in claim 1, comprises at least about 1 to 96 or more substantially identical, spatially discrete regions, each region comprising, magnetic particle array from about 2 to 500 spots.

32. A method of making the said magnetic particle array comprising;
to a magnetic array layer comprised of plurality of magnetized spots,
dispensing or microspotting reagent containing magnetic particles on the surface of the magnetic layer in spatially defined and physically addressable manner until the said magnetic array is formed.

33. A magnetic particle array comprising;
a magnet layer having individual permanently magnetized spots at a density in the range of about 1 to 100 spots per mm², each of said spots having a maximum planar dimension in the range of about 1 um to 5 mm;
immobilized at a plurality of said spots magnetic particles having diameter in the range of about 0.005 to 1,000 um, a plurality of said magnetic particles having different molecules of interest conjugated to said magnetic particles.

34. A method of using a magnetic particle array to perform an assay employing magnetic particles to which target molecules have been conjugated and a magnetic array substrate having spaced-apart magnetized spots, the method comprising:
transferring the said magnetic particles onto the surface of the magnetic array substrate with said magnetic particles being immobilized at said spaced apart magnetized spots to form a magnetic particle array with the said magnetic particles in a predetermined organization; and adding a solution comprising a composition of interest to said magnetic particle array; and determining any interaction between said composition of interest and said target molecule.

35. The said method of claim 34, wherein the step of transferring the magnetic particles onto the surface of the magnetic array substrate further comprises loading the magnetic particles into the tip of the pipette, and dispensing the magnetic particles at a predetermined location as a spot in the array.

36. The method of claim 34, wherein the composition of interest further comprises a label or a labeled complementary binding molecule is added to bind to said composition of interest.

37. The said method of claim 34, wherein the step of transferring the magnetic particles onto the surface of the magnetic array substrate further comprises transferring the magnetic particles with an electromagnetic transfer system

38. A system for screening reaction between compositions of interest, said system comprising;
vessels organized in a spatial arrangement containing magnetic particles, wherein a plurality of said vessels contain magnetic particles having different composition of interest;
a magnetic layer comprising magnetized spots spatially organized in conformance with said vessel spatial arrangement; and
a magnetic transfer device for simultaneously retrieving magnetic particles from each of the said vessels and transferring said magnetic particles to said magnetic layer in the spatial organization of said vessels.

39. A system according to claim 38, wherein said vessels are microtiter well plates

40. A system according to claim 38, further comprising a data processing unit

41. A system according to claim 38, wherein said magnetic particles have a diameter in the range of about 0.05 to 1000 um and said spots are at a density in the range of about 1 to 100 spots per mm², each of said spots having a maximum planar dimension in the range of about 1 um to 5 mm.
